SEQUENCE LISTING (109 sequences)

SEQ ID NO: 1 - DNA, 20 bp, Artificial Sequence
  Synthetic oligonucleotides
cggcgcccgagagtccacat

SEQ ID NO: 2 - DNA, 20 bp, Artificial Sequence
  Synthetic oligonucleotides
ccaggaggccggctactgcg

SEQ ID NO: 3 - DNA, 20 bp, Artificial Sequence
  Synthetic oligonucleotides
ctgggctgggctgggtgacg

SEQ ID NO: 4 - DNA, 20 bp, Artificial Sequence
  Synthetic oligonucleotides
acccgggtgagggtctgggc

SEQ ID NO: 5 - DNA, 20 bp, Artificial Sequence
  Synthetic oligonucleotides
ccaactctgccggccgcggg

SEQ ID NO: 6 - DNA, 20 bp, Artificial Sequence
  Synthetic oligonucleotides
acggcggccgcagagtgggg

SEQ ID NO: 7 - DNA, 20 bp, Artificial Sequence
  Synthetic oligonucleotides
tcgatgtcggtgcgcaggcc

SEQ ID NO: 8 - DNA, 20 bp, Artificial Sequence
  Synthetic oligonucleotides
ggcggggcgtgcaggtcgca

SEQ ID NO: 9 - DNA, 20 bp, Artificial Sequence
  Synthetic oligonucleotides
acgttggttcgaacttgcgc

SEQ ID NO: 10 - DNA, 20 bp, Artificial Sequence
  Synthetic oligonucleotides
ttccaaatctggttgaggcc

SEQ ID NO: 11 - DNA, 20 bp, Artificial Sequence
  Synthetic oligonucleotides
agacactctgctttttgaca

SEQ ID NO: 12 - DNA, 20 bp, Artificial Sequence
  Synthetic oligonucleotides
tttcctcaaattcatcaaat

SEQ ID NO: 13 - DNA, 20 bp, Artificial Sequence
  Synthetic oligonucleotides
gggtggcccaaagttccaga

SEQ ID NO: 14 - DNA, 20 bp, Artificial Sequence
  Synthetic oligonucleotides
tggtctcatctagagagcct

SEQ ID NO: 15 - DNA, 20 bp, Artificial Sequence
  Synthetic oligonucleotides
ctctgctagtctttcatagg

SEQ ID NO: 16 - DNA, 20 bp, Artificial Sequence
  Synthetic oligonucleotides
gctaaagagtccagcgtttc

SEQ ID NO: 17 - DNA, 21 bp, Artificial Sequence
  Synthetic oligonucleotides
gcaaggtcttcaaaaaactct

SEQ ID NO: 18 - DNA, 22 bp, Artificial Sequence
  Synthetic oligonucleotides
ctcaaacgtgtatggcttgtct

SEQ ID NO: 19 - DNA, 21 bp, Artificial Sequence
  Synthetic oligonucleotides
cccaaaggagacatcatagtc

SEQ ID NO: 20 - DNA, 24 bp, Artificial Sequence
  Synthetic oligonucleotides
cagtttgacagttaagacaccact

SEQ ID NO: 21 - DNA, 21 bp, Artificial Sequence
  Synthetic oligonucleotides
ataggttcctagatctccacc

SEQ ID NO: 22 - DNA, 20 bp, Artificial Sequence
  Synthetic oligonucleotides
ggcgtctgcttgttgatcac

SEQ ID NO: 23 - DNA, 23 bp, Artificial Sequence
  Synthetic oligonucleotides
aagatagccagatttgcttgttt

SEQ ID NO: 24 - DNA, 24 bp, Artificial Sequence
  Synthetic oligonucleotides
ggtccactacatacctggatggag

SEQ ID NO: 25 - DNA, 21 bp, Artificial Sequence
  Synthetic oligonucleotides
cccagtccagtcataacgctt

SEQ ID NO: 26 - DNA, 22 bp, Artificial Sequence
  Synthetic oligonucleotides
cgtgggagtacacccagttttt

SEQ ID NO: 27 - DNA, 18 bp, Artificial Sequence
  Synthetic oligonucleotides
catggagggacacgccgt

SEQ ID NO: 28 - DNA, 22 bp, Artificial Sequence
  Synthetic oligonucleotides
gtgagctctgcggccagcagct

SEQ ID NO: 29 - DNA, 21 bp, Artificial Sequence
  Synthetic oligonucleotides
agtttggtttttaaggcttta

SEQ ID NO: 30 - DNA, 21 bp, Artificial Sequence
  Synthetic oligonucleotides
taggccaaggaagacaagtcc

SEQ ID NO: 31 - DNA, 19 bp, Artificial Sequence
  Synthetic oligonucleotides
tcaagcatcttttccggaa

SEQ ID NO: 32 - DNA, 21 bp, Artificial Sequence
  Synthetic oligonucleotides
tccttaaaacggggctgggca

SEQ ID NO: 33 - DNA, 21 bp, Artificial Sequence
  Synthetic oligonucleotides
ttggcctgatagcttttaatg

SEQ ID NO: 34 - DNA, 27 bp, Artificial Sequence
  Synthetic oligonucleotides
cctcagctgcataatgaagctggggtc

SEQ ID NO: 35 - DNA, 25 bp, Artificial Sequence
  Synthetic oligonucleotides
aacaacaacaacaacaaaaaacaga

SEQ ID NO: 36 - DNA, 26 bp, Artificial Sequence
  Synthetic oligonucleotides
cctcaaaagcaggaataaaaaaaata

SEQ ID NO: 37 - DNA, 23 bp, Artificial Sequence
  Synthetic oligonucleotides
gctgtgacacatagcccaactgt

SEQ ID NO: 38 - DNA, 25 bp, Artificial Sequence
  Synthetic oligonucleotides
ggaggcaacacattctttctacaga

SEQ ID NO: 39 - DNA, 15 bp, Artificial Sequence
  Synthetic oligonucleotides
ctattaatattactg

SEQ ID NO: 40 - DNA, 16 bp, Artificial Sequence
  Synthetic oligonucleotides
cattatgtgtatgtat

SEQ ID NO: 41 - DNA, 15 bp, Artificial Sequence
  Synthetic oligonucleotides
tttatctatgttatt

SEQ ID NO: 42 - DNA, 15 bp, Artificial Sequence
  Synthetic oligonucleotides
ctaatttgaagttct

SEQ ID NO: 43 - DNA, 15 bp, Artificial Sequence
  Synthetic oligonucleotides
ttcgaacttgcgcgg

SEQ ID NO: 44 - DNA, 14 bp, Artificial Sequence
  Synthetic oligonucleotides
tagagagcctgggt

SEQ ID NO: 45 - DNA, 15 bp, Artificial Sequence
  Synthetic oligonucleotides
acaccactcccaaag

SEQ ID NO: 46 - DNA, 15 bp, Artificial Sequence
  Synthetic oligonucleotides
aggtccactacatac

SEQ ID NO: 47 - DNA, 15 bp, Artificial Sequence
  Synthetic oligonucleotides
cgttaacctggatgg

SEQ ID NO: 48 - DNA, 15 bp, Artificial Sequence
  Synthetic oligonucleotides
tgacccaagggagac

SEQ ID NO: 49 - DNA, 15 bp, Artificial Sequence
  Synthetic oligonucleotides
tggccactggccgca

SEQ ID NO: 50 - DNA, 15 bp, Artificial Sequence
  Synthetic oligonucleotides
cggcgacccctggtg

SEQ ID NO: 51 - DNA, 15 bp, Artificial Sequence
  Synthetic oligonucleotides
cgccctccagcgctg

SEQ ID NO: 52 - DNA, 15 bp, Artificial Sequence
  Synthetic oligonucleotides
cgctccgccctccag

SEQ ID NO: 53 - DNA, 17 bp, Artificial Sequence
  Synthetic oligonucleotides
tgacccaagggagaccc

SEQ ID NO: 54 - DNA, 17 bp, Artificial Sequence
  Synthetic oligonucleotides
tggccactggccgcacc

SEQ ID NO: 55 - DNA, 17 bp, Artificial Sequence
  Synthetic oligonucleotides
cggcgacccctggtgcc

SEQ ID NO: 56 - DNA, 17 bp, Artificial Sequence
  Synthetic oligonucleotides
cgccctccagcgctgcc

SEQ ID NO: 57 - DNA, 17 bp, Artificial Sequence
  Synthetic oligonucleotides
cgctccgccctccagcc

SEQ ID NO: 58 - DNA, 24 bp, Artificial Sequence
  Synthetic oligonucleotides
tgacccaagggagacggaaaccac

SEQ ID NO: 59 - DNA, 24 bp, Artificial Sequence
  Synthetic oligonucleotides
tggccactggccgcaggaaaccac

SEQ ID NO: 60 - DNA, 24 bp, Artificial Sequence
  Synthetic oligonucleotides
cggcgacccctggtgggaaacctc

SEQ ID NO: 61 - DNA, 24 bp, Artificial Sequence
  Synthetic oligonucleotides
cgccctccagcgctgggaaacctc

SEQ ID NO: 62 - DNA, 24 bp, Artificial Sequence
  Synthetic oligonucleotides
cgctccgccctccagccaaaggtc

SEQ ID NO: 63 - DNA, 15 bp, Artificial Sequence
  Synthetic oligonucleotides
ggtttttaaggcttt

SEQ ID NO: 64 - DNA, 15 bp, Artificial Sequence
  Synthetic oligonucleotides
ggggtcttggcctga

SEQ ID NO: 65 - DNA, 15 bp, Artificial Sequence
  Synthetic oligonucleotides
cataatgaagctggg

SEQ ID NO: 66 - DNA, 15 bp, Artificial Sequence
  Synthetic oligonucleotides
aggaggcaacacatt

SEQ ID NO: 67 - DNA, 15 bp, Artificial Sequence
  Synthetic oligonucleotides
attattttgcttttt

SEQ ID NO: 68 - DNA, 15 bp, Artificial Sequence
  Synthetic oligonucleotides
cattttccctcctgg

SEQ ID NO: 69 - DNA, 15 bp, Artificial Sequence
  Synthetic oligonucleotides
gtaggctacccttta

SEQ ID NO: 70 - DNA, 15 bp, Artificial Sequence
  Synthetic oligonucleotides
gaggcttgttgcttt

SEQ ID NO: 71 - DNA, 15 bp, Artificial Sequence
  Synthetic oligonucleotides
catgtatgatgttat

SEQ ID NO: 72 - DNA, 20 bp, Artificial Sequence
  Synthetic oligonucleotides
tttttggtttttaaggcttt

SEQ ID NO: 73 - DNA, 20 bp, Artificial Sequence
  Synthetic oligonucleotides
tttttggggtcttggcctga

SEQ ID NO: 74 - DNA, 20 bp, Artificial Sequence
  Synthetic oligonucleotides
tttttcataatgaagctggg

SEQ ID NO: 75 - DNA, 20 bp, Artificial Sequence
  Synthetic oligonucleotides
tttttaggaggcaacacatt

SEQ ID NO: 76 - DNA, 20 bp, Artificial Sequence
  Synthetic oligonucleotides
tttttattattttgcttttt

SEQ ID NO: 77 - DNA, 15 bp, Artificial Sequence
  Synthetic oligonucleotides
atgggggacggggca

SEQ ID NO: 78 - DNA, 15 bp, Artificial Sequence
  Synthetic oligonucleotides
ggttgagactgggtg

SEQ ID NO: 79 - DNA, 15 bp, Artificial Sequence
  Synthetic oligonucleotides
atgggggacggggca

SEQ ID NO: 80 - DNA, 15 bp, Artificial Sequence
  Synthetic oligonucleotides
aaagccttaaaaacc

SEQ ID NO: 81 - DNA, 15 bp, Artificial Sequence
  Synthetic oligonucleotides
tcaggccaagacccc

SEQ ID NO: 82 - DNA, 15 bp, Artificial Sequence
  Synthetic oligonucleotides
cccagcttcattatg

SEQ ID NO: 83 - DNA, 15 bp, Artificial Sequence
  Synthetic oligonucleotides
aatgtgttgcctcct

SEQ ID NO: 84 - DNA, 15 bp, Artificial Sequence
  Synthetic oligonucleotides
aaaaagcaaaataat

SEQ ID NO: 85 - DNA, 15 bp, Artificial Sequence
  Synthetic oligonucleotides
ccaggagggaaaatg

SEQ ID NO: 86 - DNA, 15 bp, Artificial Sequence
  Synthetic oligonucleotides
taaagggtagcctac

SEQ ID NO: 87 - DNA, 15 bp, Artificial Sequence
  Synthetic oligonucleotides
aaagcaacaagcctc

SEQ ID NO: 88 - DNA, 15 bp, Artificial Sequence
  Synthetic oligonucleotides
ataacatcatacatg

SEQ ID NO: 89 - DNA, 15 bp, Artificial Sequence
  Synthetic oligonucleotides
gatactatcttcctc

SEQ ID NO: 90 - DNA, 15 bp, Artificial Sequence
  Synthetic oligonucleotides
agactgaagaggtgc

SEQ ID NO: 91 - DNA, 15 bp, Artificial Sequence
  Synthetic oligonucleotides
cgggacggctgtgtt

SEQ ID NO: 92 - DNA, 15 bp, Artificial Sequence
  Synthetic oligonucleotides
tctgtgtgggcagca

SEQ ID NO: 93 - DNA, 15 bp, Artificial Sequence
  Synthetic oligonucleotides
aaagccttaaaaacc

SEQ ID NO: 94 - DNA, 15 bp, Artificial Sequence
  Synthetic oligonucleotides
tcaggccaagacccc

SEQ ID NO: 95 - DNA, 15 bp, Artificial Sequence
  Synthetic oligonucleotides
cccagcttcattatg

SEQ ID NO: 96 - DNA, 15 bp, Artificial Sequence
  Synthetic oligonucleotides
aatgtgttgcctcct

SEQ ID NO: 97 - DNA, 15 bp, Artificial Sequence
  Synthetic oligonucleotides
aaaaagcaaaataat

SEQ ID NO: 98 - DNA, 15 bp, Artificial Sequence
  Synthetic oligonucleotides
ccaggagggaaaatg

SEQ ID NO: 99 - DNA, 15 bp, Artificial Sequence
  Synthetic oligonucleotides
taaagggtagcctac

SEQ ID NO: 100 - DNA, 15 bp, Artificial Sequence
  Synthetic oligonucleotides
aaagcaacaagcctc

SEQ ID NO: 101 - DNA, 15 bp, Artificial Sequence
  Synthetic oligonucleotides
ataacatcatacatg

SEQ ID NO: 102 - DNA, 15 bp, Artificial Sequence
  Synthetic oligonucleotides
gatactatcttcctc

SEQ ID NO: 103 - DNA, 15 bp, Artificial Sequence
  Synthetic oligonucleotides
atgggggacggggca

SEQ ID NO: 104 - DNA, 15 bp, Artificial Sequence
  Synthetic oligonucleotides
ggttgagactgggtg

SEQ ID NO: 105 - DNA, 15 bp, Artificial Sequence
  Synthetic oligonucleotides
agactgaagaggtgc

SEQ ID NO: 106 - DNA, 15 bp, Artificial Sequence
  Synthetic oligonucleotides
cgggacggctgtgtt

SEQ ID NO: 107 - DNA, 15 bp, Artificial Sequence
  Synthetic oligonucleotides
tctgtgtgggcagca

SEQ ID NO: 108 - DNA, 15 bp, Artificial Sequence
  Synthetic oligonucleotides
gaagaagaagaagaa

SEQ ID NO: 109 - DNA, 15 bp, Artificial Sequence
  Synthetic oligonucleotides
ttcttcttcttcttc

(19) United States
(12) Patent Application Publication
(10) Pub. No.: US 2016/0201064 A1
(43) Pub. Date: Jul. 14, 2016

(54) COMPOSITIONS AND METHODS FOR MODULATING EXPRESSION OF FRATAXIN

(71) Applicant: RaNA THERAPEUTICS, INC., Cambridge, MA (US)

(72) Inventor: Fatih Ozsolak, Boston, MA (US)

(73) Assignee: RaNA Therapeutics, Inc., Cambridge, MA (US)

(21) Appl. No.: 14/911,849

(22) PCT Filed: Aug. 15, 2014
(86) PCT No.: PCT/US2014/051261

§ 371 Date: Feb. 12, 2016

Related U.S. Application Data
(60) Provisional application No. 61/866,790, filed on Aug. 16, 2013.

(51) Int. Cl. C12N 15/113

(52) U.S. Cl. CPC C12N 15/1137; C12N 2310/321; C12N 2310/322; C12N 2310/3231; C12N 2310/34; C12N 2320/30; C12N 2310/3513; C12N 2310/351

(57) ABSTRACT

Provided herein are compositions and methods for increasing Frataxin (FXN) expression. Compositions and methods for treating Friedrich's ataxia are also provided.

### CROSS-REFERENCE TO RELATED APPLICATIONS

This application claims the benefit under 35 U.S.C. §119(e) of U.S. Provisional Application No. 61/866,790, entitled “COMPOSITIONS AND METHODS FOR MODULATING EXPRESSION OF FRATAXIN”, filed Aug. 16, 2013, the contents of which are incorporated herein by reference in its entirety.

### FIELD OF THE INVENTION

The invention relates in part to compositions and methods for modulating gene expression.

### BACKGROUND OF THE INVENTION

Friedreich's ataxia (FRDA) is a rare recessive inherited disease characterized by progressive degeneration of the spinal cord and peripheral nerve tissue. Symptoms resulting from this nervous system damage include muscle weakness, loss of coordination, vision and hearing impairment, speech problems, scoliosis, diabetes, and several heart disorders. Symptoms typically begin between ages of 5 and 15 years and first present as difficulty walking (gait ataxia). As the disease progresses, other symptoms develop, such as speech slurring, hearing loss, and vision loss. Various forms of heart disease often accompany FRDA, including hypertrophic cardiomyopathy, myocardial fibrosis, and cardiac failure. Approximately, ten percent of those affected by FRDA develop diabetes. Symptom progression varies between individuals, but generally within 10 to 20 years from disease onset, the person is wheelchair bound and may eventually become completely incapacitated. FRDA can lead to early death, often as a result of heart disease associated with FRDA. Reduced expression of Frataxin (FXN) is thought to be a cause of Friedreich's ataxia (FRDA).

### SUMMARY OF THE INVENTION

Aspects of the invention relate to oligonucleotides and related methods and compositions for modulating FXN expression. Aspects of the invention are based on the identification of oligonucleotides that are useful for selectively upregulating FXN expression in cells. In some embodiments, oligonucleotides provided herein that upregulate FXN expression are complementary to certain regions of the sense strand of an FXN gene (e.g., a human FXN gene). In some embodiments, oligonucleotides provided herein that upregulate FXN expression are complementary to certain regions of an FXN mRNA (e.g., a human FXN mRNA). In some embodiments, oligonucleotides are provided for increasing levels of FXN mRNA in cells from a patient with FRDA. In some embodiments, oligonucleotides are provided for increasing levels of FXN protein in cells from a patient with FRDA. In some embodiments, the methods and compositions are useful for the treatment and/or prevention (e.g., reducing the risk or delaying the onset) of FRDA. In some embodiments, oligonucleotides are provided that are complementary to a FXN target (e.g., FXN mRNA) and thereby cause upregulation of the FXN.

In some embodiments, oligonucleotides are provided with chemistries suitable for delivery, hybridization and stability within cells. Furthermore, in some embodiments, oligonucleotide chemistries are provided that are useful for controlling the pharmacokinetics, biodistribution, bioavailability and/or efficacy of the oligonucleotides.

Aspects of the invention relate to methods for increasing expression of Frataxin (FXN) in cells. In some embodiments, the methods involve delivering to a cell an oligonucleotide comprising at least 8 nucleotides of a nucleotide sequence as set forth in Table 2 or 3, thereby increasing FXN expression in the cell. In some embodiments, prior to the step of delivering, the cell has a higher level of histone H3 K27 or K9 methylation at the FXN gene compared with an appropriate control level of histone H3 K27 or K9 methylation. In some embodiments, the cell comprises an FXN gene encoding in its first intron a GAA repeat of between 10-2000 units. In certain embodiments, the cell is in a subject having Friedreich's ataxia.

In some embodiments, an oligonucleotide provided herein is a single stranded oligonucleotide. In certain embodiments, an oligonucleotide provided herein comprises at least one modified internucleoside linkage. In some embodiments, an oligonucleotide provided herein comprises at least one modified nucleotide. In certain embodiments, an oligonucleotide provided herein comprises at least one nucleotide comprising a 2′ O-methyl. In some embodiments, an oligonucleotide provided herein comprises at least one ribonucleotide, at least one deoxyribonucleotide, at least one 2′-fluoro-deoxyribonucleotide or at least one bridged nucleotide. In certain embodiments, the bridged nucleotide is a LNA nucleotide, a cEt nucleotide or a ENA modified nucleotide. In some embodiments, each nucleotide of the oligonucleotide is a LNA nucleotide. In certain embodiments, the oligonucleotide is mixmer. In some embodiments, the oligonucleotide comprises alternating deoxyribonucleotides and 2′-fluoro-deoxyribonucleotides, 2′-O-methyl nucleotides, or bridged nucleotides. In certain embodiments, the oligonucleotide comprises a sequence as set forth in Table 2 or Table 3. In some embodiments, the oligonucleotide is 8 to 50 nucleotides in length. In certain embodiments, the oligonucleotide consists of a sequence as set forth in Table 2 or Table 3.

According to some aspects of the invention, an oligonucleotide of 8 to 50 nucleotides in length is provided that comprising at least 8 consecutive nucleotides of a nucleotide sequence as set forth in Table 2 or 3. In certain embodiments, the oligonucleotide comprises a sequence as set forth in Table 2 or Table 3. In some embodiments, the oligonucleotide comprises a fragment of at least 8 nucleotides of a nucleotide sequence as set forth in Table 2 or 3. In certain embodiments, the oligonucleotide consists of a sequence as set forth in Table 2 or Table 3.

In some aspects of the invention, compositions are provided that comprise one or more oligonucleotides disclosed herein. In some embodiments, compositions are provided that comprise a plurality of oligonucleotides, in which each of at least 75% of the oligonucleotides comprise or consist of a nucleotide sequence as set forth in Table 2 or 3. In some embodiments, the oligonucleotide is complexed with a monovalent cation (e.g., Li+, Na+, K+, Cs+). In some embodiments, the oligonucleotide is in a lyophilized form. In some embodiments, the oligonucleotide is in an aqueous solution. In some embodiments, the oligonucleotide is provided, combined or mixed with a carrier (e.g., a pharmaceutically acceptable carrier). In some embodiments, the oligonucleotide is provided in a buffered solution. In some embodiments, the oligonucleotide is conjugated to a carrier. In some aspects of the invention, kits are provided that comprise a container housing the composition.

The details of one or more embodiments of the invention are set forth in the description below. Other features or advantages of the present invention will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 is a graph depicting FXN mRNA levels after the targeting of frataxin (FXN) with mixmer oligonucleotides.

FIG. 2 is a graph depicting FXN protein levels after treatment of cells with FXN UTR-targeting oligos or with other oligonucleotides identified as being capable of regulating FXN mRNA.

FIG. 3 is a graph depicting that treatment of cells with a combination of FXN RNA stabilizing oligonucleotides and transcriptional/post-transcriptional oligos increased FXN mRNA levels.

FIG. 4 is a graph depicting increased FXN mRNA levels 2-3 days post-treatment in cell treated with oligonucleotides that target the 5′UTR, the 3′UTR, or the internal region of the human FXN. The steady-state mRNA levels of the oligos approached the levels of FXN mRNA in the GM0321B normal fibroblast cells. X-axis lists oligonucleotide numbers and control identifiers.

FIG. 5A is a graph depicting result of an evaluation of cytoxicity (CTG) at two days of treatment. Treatment of the FRDA patient cell line GM03816 with oligos did not result in cytotoxicity during day 2 of oligo treatment at 100 and 400 nM. X-axis lists oligonucleotide numbers and control identifiers.

FIG. 5B is a graph depicting an results of an evaluation of cytoxcity (CTG) after three days of treatment. Treatment of the FRDA patient cell line GM03816 with oligos did not result in cytotoxicity during day 3 of oligo treatment at 100 and 400 nM.

FIG. 6 is a graph showing FXN mRNA upregulation in cells treated with oligo 329.

### DETAILED DESCRIPTION OF THE INVENTION

Aspects of the invention provided herein relate to identification of oligonucleotides useful for upregulating frataxin (FXN). In some embodiments, oligonucleotides provided herein were developed based on assessment of various potential target regions of the FXN gene. In some embodiments, it has been discovered that oligonucleotides complementary to certain regions of the sense strand of the human FXN gene cause upregulation of FXN when delivered to cells. Accordingly, in some embodiments, oligonucleotides provided herein are complementary to regions of the sense strand of an FXN gene (e.g., a human FXN gene). In some embodiments, oligonucleotides provided herein are complementary to regions of an FXN mRNA (e.g., a human FXN mRNA). In some embodiments, oligonucleotides are provided that, when administered to a cell or a subject, result in upregulation of FXN. In some embodiments, oligonucleotides disclosed herein may specifically hybridize in cells with an FXN mRNA bringing about increase levels of the mRNA. However, in some embodiments, oligonucleotides disclosed herein may specifically hybridize in cells with a DNA strand (e.g., the sense strand) of an FXN gene.

As used herein, the term “FXN gene” refers to a genomic region that encodes FXN protein and/or controls the transcription of FXN mRNA. Thus, the term encompasses coding sequences and exons as well as any non-coding elements, e.g., promoters, enhancers, silencers, introns, and 5′ and 3′ untranslated regions. An FXN gene may include flanking sequences 5′ and/or 3′ to a known annotated FXN open reading frame, e.g., 1 Kb, 2 Kb, 3 Kb, 4 Kb, 5 Kb, 6 Kb, 7 Kb, 8 Kb, 9 Kb, or 10 Kb or more flanking the 5′ and/or 3′ end of a known annotated FXN open reading frame. In some embodiments, a FXN gene may be a human FXN gene (see, e.g., NCBI Gene ID: 2395, located on chromosome 9). In some embodiments, a FXN gene may be a corresponding homolog of a FXN gene in a different species (e.g., a mouse FXN encoded by a mouse FXN gene such as NCBI Gene ID: 14297).

As used herein, mRNA includes pre-mRNA and mature mRNA. FXN human and mouse mRNA and corresponding protein sequences are known in the art. Examples of human FXN mRNA sequences are provided at NCBI accession numbers: NM_000144.4, NM_001161706.1, NM_181425.2 (SEQ ID NOs: 110-112). Examples of mouse FXN mRNA sequences are provided at NCBI accession numbers. NM_008044.2 (SEQ ID NO: 113). Examples of human FXN protein sequences are provided at NCBI accession numbers: NP_000135.2, NP_001155178.1, NP_852090.1. Examples of mouse FXN protein sequences are provided at NCBI accession numbers NP_032070.1.

In some embodiments, oligonucleotides disclosed herein may specifically hybridize in cells with a non-coding RNA at least portion of which is encoded by genomic sequences residing within the FXN gene, resulting in increased level of FXN mRNA, e.g., resulting from upregulation of FXN mRNA transcription. In some embodiments, oligonucleotides disclosed herein may specifically hybridize in cells with a non-coding RNA transcribed from a genomic location that is contained, in part or in whole, within a FXN gene (e.g., a non-coding RNA that is transcribed from a genomic location contained, in part or in whole, within the boundaries of a FXN gene) or nearby a FXN, e.g., within 20 Kb, 15 Kb, 10 Kb, 5 Kb, 2 Kb or 1 Kb of a FXN gene.

### Methods for Modulating FXN Gene Expression

In one aspect, the invention relates to methods for modulating FXN gene expression in a cell (e.g., a cell for which FXN levels are reduced) for research purposes (e.g., to study the function of the gene in the cell). In another aspect, the invention relates to methods for modulating gene expression in a cell (e.g., a cell for which FXN levels are reduced) for gene or epigenetic therapy. The cells can be in vitro, ex vivo, or in vivo (e.g., in a subject who has a disease resulting from reduced expression or activity of FXN, e.g., Friedreich's ataxia). In some embodiments, methods for modulating gene expression in a cell comprise delivering an oligonucleotide as described herein.

It is understood that any reference to uses of compounds throughout the description contemplates use of the compound in preparation of a pharmaceutical composition or medicament for use in the treatment of condition or a disease (e.g., Friedreich's ataxia) associated with decreased levels or activity of FXN. Thus, as one non-limiting example, this aspect of the invention includes use of such oligonucleotides in the preparation of a medicament for use in the treatment of disease, wherein the treatment involves upregulating expression of FXN.

In some embodiments, the method comprises contacting a cell with an oligonucleotide described herein, e.g., an oligonucleotide having a region of complementarity with at least 5 nucleotides of a FXN gene or mRNA region (e.g., SEQ ID NOs: 110-113), thereby increasing FXN expression in the cell. In some embodiments, the method comprises contacting a cell having a lower level of FXN expression compared to an appropriate control level of FXN expression with an oligonucleotide described herein, e.g., an oligonucleotide having a region of complementarity with at least 5 nucleotides of a FXN gene or mRNA region (e.g., SEQ ID NOs: 110-113), thereby increasing FXN expression in the cell. In some embodiments, it is contemplated that the cell may be contacted with more than one oligonucleotide that targets multiple regions of a FXN gene or mRNA, e.g., a first oligonucleotide that targets a first region of a FXN gene or mRNA and a second oligonucleotide that targets a second region of a FXN gene or mRNA. In some embodiments, it is contemplated that the cell may be contacted with more than one oligonucleotide, e.g., a first oligonucleotide that targets a first region of an FXN gene or mRNA and a second oligonucleotide that targets a second region of an FXN gene or mRNA, resulting in upregulation of FXN expression.

In some embodiments, a cell having a lower level of FXN expression compared to an appropriate control level of FXN expression has a level of FXN expression that is at least 5%, 10%, 20%, 30%, 40%, 50%, 60%, 70%, 80%, 90%, 100%, 200%, 300%, 400%, 500% or more lower than an appropriate control level of FXN expression. A level of FXN expression may be determined using any suitable assay known in the art (see, e.g., Molecular Cloning: A Laboratory Manual, J. Sambrook, et al., eds., Third Edition, Cold Spring Harbor Laboratory Press, Cold Spring Harbor, N.Y., 2001; Current Protocols in Molecular Biology, Current Edition, John Wiley & Sons, Inc., New York; and Current Protocols in Protein Production, Purification, and Analysis, Current Edition, John Wiley & Sons, Inc., New York). The FXN expression level may be an mRNA level or a protein level. The sequences of FXN mRNAs and proteins are well-known in the art and are provided herein and can be used to design suitable reagents and assays for measuring an FXN expression level.

In some embodiments, an appropriate control level of FXN expression may be, e.g., a level of FXN expression in a cell, tissue or fluid obtained from a healthy subject or population of healthy subjects. As used herein, a healthy subject is a subject that is apparently free of disease and has no history of disease, e.g., no history of Friedreich's ataxia. In some embodiments, an appropriate control level of is a level of FXN expression in a cell from a subject that does not have Friedreich's ataxia or a level of FXN expression in a population of cells from a population of subjects that do not have Friedreich's ataxia. In some embodiments, the subject or population of subjects that do not have Friedreich's ataxia are subjects that have a FXN gene that contains less than 20, 19, 18, 17, 16, 15, 14, 13, 12, 11, or 10 GAA repeat units in the first intron. In some embodiments, oligonucleotides provided herein for increasing FXN expression are not complementary to a nucleic acid sequence within the first intron of FXN. In some embodiments, when a level of FXN expression is elevated or increased compared to a control level of FXN, an appropriate control level of FXN may be a level of FXN expression in a cell, tissue, or subject to which an oligonucleotide has not been delivered or to which a negative control has been delivered (e.g., a scrambled oligo, a carrier, etc.).

In some embodiments, an appropriate control level of FXN expression may be a predetermined level or value, such that a control level need not be measured every time. The predetermined level or value can take a variety of forms. It can be single cut-off value, such as a median or mean. It can be established based upon comparative groups, such as where one defined group is known have Friedriech's ataxia and another defined group is known to not have Friedriech's ataxia. It can be a range, for example, where the tested population is divided equally (or unequally) into groups, such as a group of subjects having a high number of GAA repeats in the first intron of FXN (e.g., over 1000 GAA repeats), a group of subjects having a moderate number of GAA repeats (e.g., from 20-1000 GAA repeats) and a group of subjects having a low number of GAA repeats (e.g., less than 20 GAA repeats).

The predetermined value can depend upon the particular population selected. Accordingly, the predetermined values selected may take into account the category in which a subject falls. Appropriate ranges and categories can be selected with no more than routine experimentation by those of ordinary skill in the art.

In some embodiments, a cell having a lower level of FXN expression compared to an appropriate control level of FXN expression is a cell that has a higher level of histone H3 K27 or K9 methylation at the FXN gene compared with an appropriate control level of histone H3 K27 or K9 methylation. An appropriate control level of histone H3 K27 or K9 methylation may be, e.g., a level of histone H3 K27 or K9 methylation in a cell, tissue or fluid obtained from a healthy subject or population of healthy subjects, such as a subject or subjects that do not have Friedreich's ataxia. A level of H3 K27 or K9 methylation expression may be determined using any suitable assay known in the art. Examples of assays for detecting histone methylation levels include, but are not limited to, immunoassays such as Western blot, immunohistochemistry and ELISA assays. Such assays may involve a binding partner, such as an antibody, that specifically binds to a methylated or unmethylated histone. Antibodies that recognize specific methylation patterns on histones are known in the art and available from commercial vendors (see, e.g., AbCam and Millipore).

In some embodiments, a cell having a lower level of FXN expression compared to an appropriate control level of FXN expression is a cell that comprises an FXN gene encoding in its first intron a GAA repeat of between 10-2000, 15-2000, 20-2000, 30-2000, 40-2000, 50-2000, 100-2000, 10-1000, 15-1000, 20-1000, 30-1000, 40-1000, 50-1000, or 100-1000 units. The number of GAA repeats may be determined using any method known in the art, e.g., sequencing-based assays or probe-based assays.

In some embodiments, a cell having a lower level of FXN expression compared to an appropriate control level of FXN expression is a cell obtained from or in a subject having Friedreich's ataxia. A subject having Friedreich's ataxia can be identified, e.g., by the number of GAA repeats present in the first intron of an FXN gene of the subject and/or by other diagnostic criteria or symptoms known in the art. Symptoms of Friedreich's ataxia include, but are not limited to, muscle weakness in the arms and legs, loss of coordination, vision impairment, hearing impairment, slurred speech, curvature of the spine, high plantar arches, diabetes, and/or heart disorders (e.g., cardiomegaly, atrial fibrillation, tachycardia and hypertrophic cardiomyopathy). A physical examination of eye movements, deep tendon reflexes, extensor plantar responses, and cardiac sounds may aid in diagnosis of a subject suspected of having Friedreich's ataxia. A genetic test, e.g., a PCR-based test or other nucleic acid based assay, may be used to identify a subject having expanded GAA triplet repeats in the first intron of FXN.

As used herein, increasing FXN expression in a cell includes a level of FXN expression that is, e.g., 1%, 5%, 10%, 20%, 30%, 40%, 50%, 60%, 70%, 80%, 90%, 100%, 150%, 200%, 300%, 400%, 500% or more above an appropriate control level of FXN. The appropriate control level may be a level of FXN expression in a cell that has not been contacted with an oligonucleotide as described herein. In some embodiments, increasing FXN expression in a cell includes increasing a level of FXN expression to within 50%, 40%, 30%, 20%, 10%, 5%, 4%, 3%, 2%, 1%, or less of a level of FXN expression in a cell from a healthy subject or a population of cells from a population of healthy subjects, e.g., subjects that do not have Friedreich's ataxia. For example, it may be desirable to increase an FXN expression level in a cell obtained from or in subject having Friedreich's ataxia such that the level of FXN expression is approximately the same as the level of FXN expression in a cell obtained from or in a subject who is healthy (e.g., not having Friedreich's ataxia). However, it is to be understood that the level of FXN expression level in a cell obtained from or in subject having Friedreich's ataxia may be increased to a level that is higher than the level of FXN expression in a cell obtained from or in a subject who is healthy.

In another aspect of the invention, methods comprise administering to a subject (e.g. a human) a composition comprising an oligonucleotide as described herein to increase FXN protein levels in the subject. In some embodiments, the increase in protein levels is at least 5%, 10%, 20%, 30%, 40%, 50%, 60%, 70%, 80%, 90%, 100%, 200%, or more, higher than the amount of a protein in the subject before administering the oligonucleotide.

In another aspect of the invention provides methods of treating a condition (e.g., Friedreich's ataxia) associated with decreased levels of expression of FXN in a subject, the method comprising administering an oligonucleotide as described herein.

A subject can include a non-human mammal, e.g. mouse, rat, guinea pig, rabbit, cat, dog, goat, cow, or horse. In preferred embodiments, a subject is a human. Oligonucleotides have been employed as therapeutic moieties in the treatment of disease states in animals, including humans. Oligonucleotides can be useful therapeutic modalities that can be configured to be useful in treatment regimens for the treatment of cells, tissues and animals, especially humans.

For therapeutics, an animal, preferably a human, suspected of having Friedreich's ataxia is treated by administering an oligonucleotide in accordance with the invention. For example, in one non-limiting embodiment, the methods comprise the step of administering to the animal in need of treatment, a therapeutically effective amount of an oligonucleotide as described herein.

### Oligonucleotides for Modulating Expression of FXN

As described herein, oligonucleotides have been designed that are complementary to certain regions of a FXN gene, a FXN mRNA or a non-coding RNA expressed from within the FXN gene, collectively referred to as “FXN targets”. In some embodiments, oligonucleotides have been designed that are complementary to certain regions of a sense strand of a FXN gene. Oligonucleotides have been identified that are capable of upregulating FXN expression levels.

Thus, it is contemplated herein that oligonucleotides tiling a whole FXN gene or mRNA sequence can be used to identify sites important for RNA stability/quantity and to identify therapeutically relevant oligonucleotides that upregulate FXN expression levels.

In some embodiments, oligonucleotides may be identified that function to alter RNA stability or the ability of RNA to be translated by other mechanisms beyond RNA degradation. In some embodiments, translation of RNA to protein is regulated by certain RNA regions such as sequences near the 5′ end called Shine Dalgarno/Kozak sequences and internal ribosome entry sites that regulate interaction with ribosomes. In some embodiments, other sequence and structure elements that favor and disfavor RNA translation may be targeted. In some embodiments, oligonucleotides targeting such regulatory regions on FXN transcripts may be used to alter steady-state FXN mRNA and associated protein levels. In some embodiments, oligonucleotides that target RNA regulatory regions such as riboswitch-like structures that has an effect on RNA transcription may be identified.

In some embodiments, another mechanism to alter transcript levels is preventing premature transcription termination. In some embodiments, transcripts, such as FXN mRNA transcripts, undergo premature transcript termination within the gene body (such as in an internal exon or intron) yielding short and unstable transcripts. Such events can be modulated by identifying corresponding regulatory regions and using oligonucleotides to alter availability of such sites at both the DNA and RNA level, or by using oligonucleotides to block the RNA cleavage sites resulting in premature termination.

In one aspect of the invention, oligonucleotides are provided for modulating expression of FXN in a cell. In some embodiments, expression of FXN is upregulated or increased. In some embodiments, oligonucleotides are provided that have a region of complementarity to at least 5 nucleotides of a FXN target (e.g., SEQ ID NOs: 110-113).

The oligonucleotide may be single stranded or double stranded. Single stranded oligonucleotides may include secondary structures, e.g., a loop or helix structure. In some embodiments, the oligonucleotide comprises at least one modified nucleotide or modified internucleoside linkage as described herein.

The oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches.

The oligonucleotide may have a sequence that has less than a threshold level of sequence identity with every sequence of nucleotides, of equivalent length, that map to a genomic position encompassing or in proximity to an off-target gene. For example, an oligonucleotide may be designed to ensure that it does not have a sequence that maps to genomic positions encompassing or in proximity with all known genes (e.g., all known protein coding genes) other than a FXN target. The threshold level of sequence identity may be 50%, 60%, 70%, 80%, 85%, 90%, 95%, 99% or 100% sequence identity.

The oligonucleotide may have a sequence that is has greater than 30% G-C content, greater than 40% G-C content, greater than 50% G-C content, greater than 60% G-C content, greater than 70% G-C content, or greater than 80% G-C content. The oligonucleotide may have a sequence that has up to 100% G-C content, up to 95% G-C content, up to 90% G-C content, or up to 80% G-C content. In some embodiments in which the oligonucleotide is 8 to 10 nucleotides in length, all but 1, 2, 3, 4, or 5 of the nucleotides of the complementary sequence of a FXN target are cytosine or guanosine nucleotides. In some embodiments, the sequence of the target to which the oligonucleotide is complementary comprises no more than 3 nucleotides selected from adenine and uracil.

In some embodiments, an oligonucleotide may be complementary to a FXN target of a different species (e.g., a mouse, rat, rabbit, goat, monkey, etc.). In some embodiments, an oligonucleotide may be complementary to a human FXN target and also be complementary to a mouse FXN target. For example, an oligonucleotide may be complementary to a sense strand of the human FXN gene, and complementary to a sense strand of the mouse FXN gene. In another example, the oligonucleotide may be complementary to a sequence as set forth in SEQ ID NOs: 110-112, which are human FXN mRNAs, and also be complementary to a sequence as set forth in SEQ ID NO: 113, which is a mouse FXN mRNA. Oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of the oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a FXN target. In some embodiments, the region of complementarity is complementary with at least 5, at least 6, at least 7, or at least 8 consecutive nucleotides of a FXN target. In some embodiments the sequence of the oligonucleotide is based on an RNA sequence that binds to a FXN target, or a portion thereof, said portion having a length of from 5 to 40 contiguous base pairs, or about 8 to 40 bases, or about 5 to 15, or about 5 to 30, or about 5 to 40 bases, or about 5 to 50 bases.

Complementary, as the term is used in the art, refers to the capacity for precise pairing between two nucleotides. For example, if a nucleotide at a certain position of an oligonucleotide is capable of hydrogen bonding with a nucleotide at the same position of a FXN target then the oligonucleotide and the target are considered to be complementary to each other at that position. The oligonucleotide and the target are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and the FXN target. For example, if a base at one position of an oligonucleotide is capable of hydrogen bonding with a base at the corresponding position of a FXN target, then the bases are considered to be complementary to each other at that position. 100% complementarity is not required.

The oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a FXN target. In some embodiments the oligonucleotide may contain 1, 2 or 3 base mismatches compared to the portion of the consecutive nucleotides of a FXN target. In some embodiments the oligonucleotide may have up to 3 mismatches over 15 bases, or up to 2 mismatches over 10 bases.

It is understood in the art that a complementary nucleotide sequence need not be 100% complementary to that of its target to be specifically hybridizable or specific for a target molecule. In some embodiments, a complementary nucleic acid sequence for purposes of the present disclosure is specifically hybridizable or specific for the target molecule when binding of the sequence to the target molecule (e.g., a FXN target) causes a desirable outcome, e.g., upregulation of FXN, and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50 or more nucleotides in length. In a preferred embodiment, the oligonucleotide is 8 to 30 nucleotides in length.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C,

U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, GC content of the oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

It is to be understood that any oligonucleotide provided herein can be excluded.

In some embodiments, it has been found that oligonucleotides disclosed herein may increase expression of FXN mRNA by at least about 50% (i.e. 150% of normal or 1.5 fold), or by about 2 fold to about 5 fold. In some embodiments, expression may be increased by at least about 15 fold, 20 fold, 30 fold, 40 fold, 50 fold, 100 fold, 200 fold, 500 fold, 1000 fold or any range between any of the foregoing numbers.

### Oligonucleotide Modifications

The oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not mediate cleavage of a FXN mRNA, do not recruit an RNase, do not mediate alternative splicing; are not immune stimulatory; are nuclease resistant; have improved cell uptake compared to unmodified oligonucleotides; are not toxic to cells or mammals; and/or have improved endosomal exit.

Any of the oligonucleotides disclosed herein may be linked to one or more other oligonucleotides disclosed herein by a linker, e.g., a cleavable linker.

Oligonucleotides of the invention can be stabilized against nucleolytic degradation such as by the incorporation of a modification, e.g., a nucleotide modification. For example, nucleic acid sequences of the invention include a phosphorothioate at least the first, second, or third internucleoside linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom.

Any of the modified chemistries or formats of oligonucleotides described herein can be combined with each other, and that one, two, three, four, five, or more different types of modifications can be included within the same molecule.

In some embodiments, an oligonucleotide may comprise one or more modified nucleotides (also referred to herein as nucleotide analogs). In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the oligonucleotide has one or more nucleotide analogues. For example, the oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Preferably the oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the oligonucleotide comprises at least one nucleotide modified at the 2′ position of the sugar, preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than 2′-deoxyoligonucleotides against a given target.

A number of nucleotide modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-O,4′-C-ethylene-bridged nucleic acids.

Examples of LNAs are described in WO/2008/043753 and include compounds of the following general formula.

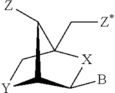

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

In some embodiments, the LNA used in the oligonucleotides described herein comprises at least one LNA unit according any of the formulas

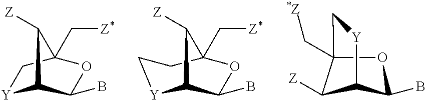

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the invention comprises internucleoside linkages selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown below:

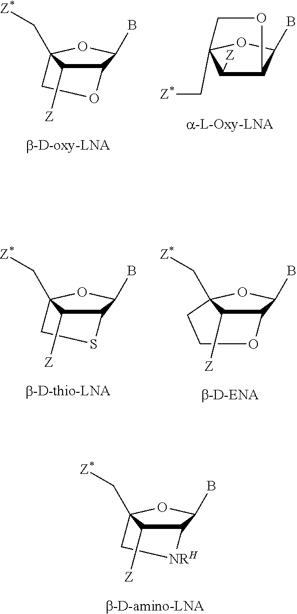

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′—O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys.

Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, oligonucleotide modification includes modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of the oligonucleotide. In some embodiments, the oligonucleotide comprises a biotin moiety conjugated to the 5′ nucleotide.

In some embodiments, the oligonucleotide comprises locked nucleic acids (LNA), ENA modified nucleotides, 2′-O-methyl nucleotides, or 2′-fluoro-deoxyribonucleotides. In some embodiments, the oligonucleotide comprises alternating deoxyribonucleotides and 2′-fluoro-deoxyribonucleotides. In some embodiments, the oligonucleotide comprises alternating deoxyribonucleotides and 2′-O-methyl nucleotides. In some embodiments, the oligonucleotide comprises alternating deoxyribonucleotides and ENA modified nucleotides. In some embodiments, the oligonucleotide comprises alternating deoxyribonucleotides and locked nucleic acid nucleotides. In some embodiments, the oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the oligonucleotide comprises phosphorothioate internucleoside linkages. In some embodiments, the oligonucleotide comprises phosphorothioate internucleoside linkages between at least two nucleotides. In some embodiments, the oligonucleotide comprises phosphorothioate internucleoside linkages between all nucleotides.

It should be appreciated that the oligonucleotide can have any combination of modifications as described herein.

In some embodiments, an oligonucleotide described herein may be a mixmer or comprise a mixmer sequence pattern. The term mixmer' refers to oligonucleotides which comprise both naturally and non-naturally occurring nucleotides or comprise two different types of non-naturally occuring nucleotides. Mixmers are generally known in the art to have a higher binding affinity than unmodified oligonucleotides and may be used to specifically bind a target molecule, e.g., to block a binding site on the target molecule. Generally, mixmers do not recruit an RNAse to the target molecule and thus do not promote cleavage of the target molecule.

In some embodiments, the mixmer comprises or consists of a repeating pattern of nucleotide analogues and naturally occurring nucleotides, or one type of nucleotide analogue and a second type of nucleotide analogue. However, it is to be understood that the mixmer need not comprise a repeating pattern and may instead comprise any arrangement of nucleotide analogues and naturally occurring nucleotides or any arrangement of one type of nucleotide analogue and a second type of nucleotide analogue. The repeating pattern, may, for instance be every second or every third nucleotide is a nucleotide analogue, such as LNA, and the remaining nucleotides are naturally occurring nucleotides, such as DNA, or are a 2′ substituted nucleotide analogue such as 2′MOE or 2′ fluoro analogues, or any other nucleotide analogues described herein. It is recognised that the repeating pattern of nucleotide analogues, such as LNA units, may be combined with nucleotide analogues at fixed positions—e.g. at the 5′ or 3′ termini.

In some embodiments, the mixmer does not comprise a region of more than 5, more than 4, more than 3, or more than 2 consecutive naturally occurring nucleotides, such as DNA nucleotides, in some embodiments, the mixmer comprises at least a region consisting of at least two consecutive nucleotide analogues, such as at least two consecutive LNAs. In some embodiments, the mixmer comprises at least a region consisting of at least three consecutive nucleotide analogue units, such as at least three consecutive LNAs.

In some embodiments, the mixmer does not comprise a region of more than 7, more than 6, more than 5, more than 4, more than 3, or more than 2 consecutive nucleotide analogues, such as LNAs. It is to be understood that the LNA units may be replaced with other nucleotide analogues, such as those referred to herein.

In some embodiments, the mixmer comprises at least one nucleotide analogue in one or more of six consecutive nucleotides. The substitution pattern for the nucleotides may be selected from the group consisting of Xxxxxx, xXxxxx, xxXxxx, xxxXxx, xxxxXx and xxxxxX, wherein “X” denotes a nucleotide analogue, such as an LNA, and “x” denotes a naturally occuring nucleotide, such as DNA or RNA.

In some embodiments, the mixmer comprises at least two nucleotide analogues in one or more of six consecutive nucleotides. The substitution pattern for the nucleotides may be selected from the group consisting of XXxxxx, XxXxxx, XxxXxx, XxxxXx, XxxxxX, xXXxxx, xXxXxx, xXxxXx, xXxxxX, xxXXxx, xxXxXx, xxXxxX, xxxXXx, xxxXxX and xxxxXX, wherein “X” denotes a nucleotide analogue, such as an LNA, and “x” denotes a naturally occuring nucleotide, such as DNA or RNA. In some embodiments, the substitution pattern for the nucleotides may be selected from the group consisting of XxXxxx, XxxXxx, XxxxXx, XxxxxX, xXxXxx, xXxxXx, xXxxxX, xxXxXx, xxXxxX and xxxXxX. In some embodiments, the substitution pattern is selected from the group consisting of XXxXxx, xXxxXx, xXxxxX, xxXxXx, xxXxxX and xxxXxX. In some embodiments, the substitution pattern is selected from the group consisting of xXxXxx, xXxxXx and xxXxXx. In some embodiments, the substitution pattern for the nucleotides is xXxXxx.

In some embodiments, the mixmer comprises at least three nucleotide analogues in one or more of six consecutive nucleotides. The substitution pattern for the nucleotides may be selected from the group consisting of XXXxxx, xXXXxx, xxXXXx, xxxXXX, XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx, wherein “X” denotes a nucleotide analogue, such as an LNA, and “x” denotes a naturally occuring nucleotide, such as DNA or RNA. In some embodiments, the substitution pattern for the nucleotides is selected from the group consisting of XXxXxx, XXxxXx, XXxxxX, xXXxXx, xXXxxX, xxXXxX, XxXXxx, XxxXXx, XxxxXX, xXxXXx, xXxxXX, xxXxXX, xXxXxX and XxXxXx. Luxonze embodiments, the substitution pattern for the nucleotides is selected from the group consisting of xXXxXx, xXXxxX, xxXXxX, xXxXXx, xXxxXX, xxXxXX and xXxXxX. some embodiments, the substitution pattern for the nucleotides is xXxXxX or XxXxXx. some embodiments, the substitution pattern for the nucleotides is xXxXxX.

In some embodiments, the mixmer comprises at least four nucleotide analogues in one or more of six consecutive nucleotides, The substitution pattern for the nucleotides may be selected from the group consisting of xXXXX, xXxXXX, xXXxXX, xXXXxX, xXXXXx, XxxXXX, XxXxXX, XxXXxX, XxXXXx, XXxxXX, XXxXxX, XXxXXx, XXXxxX, XXXxXx and XXXXxx, wherein “X” denotes a nucleotide analogue, such as an LNA, and “x” denotes a naturally occuring nucleotide, such as DNA or RNA.

In some embodiments, the mixmer comprises at least five nucleotide analogues in one or more of six consecutive nucleotides. The substitution pattern for the nucleotides may be selected from the group consisting of xXXXXX, XxXXXX, XXxXXX, XXXxXX, XXXXxX and XXXXXx, wherein “X” denotes a nucleotide analogue, such as an LNA, and “x” denotes a naturally occuring nucleotide, such as DNA or RNA.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

In some embodiments, the mixmer contains a modified nucleotide, e.g., an LNA, at the 5′ end. In some embodiments, the mixmer contains a modified nucleotide, e.g., an LNA, at the first two positions, counting from the 5′ end.

In some embodiments, the mixmer is incapable of recruiting RNAseH. Oligonucleotides that are incapable of recruiting RNAseH are well known in the literature, in example see WO2007/112754, WO2007/112753, or PCT/DK2008/000344. Mixmers may be designed to comprise a mixture of affinity enhancing nucleotide analogues, such as in non-limiting example LNA nucleotides and 2′-O-methyl nucleotides. In some embodiments, the mixmer comprises modified internucleoside linkages (e.g., phosphorothioate internucleoside linkages or other linkages) between at least two, at least three, at least four, at least five or more nucleotides.

A mixmer may be produced using any method known in the art or described herein. Representative U.S. patents, U.S. patent publications, and PCT publications that teach the preparation of mixmers include U.S. patent publication Nos. US20060128646, US20090209748 US20090298916, US20110077288 and US20120322851 and U.S. Pat. No. 7,687,617.

In some embodiments, the oligonucleotide is a gapmer. A gapmer oligonucleotide generally has the formula 5′-X-Y-Z-3′, with X and Z as flanking regions around a gap region Y. In some embodiments, the Y region is a contiguous stretch of nucleotides, e.g., a region of at least 6 DNA nucleotides, which are capable of recruiting an RNAse, such as RNAseH. Without wishing to be bound by theory, it is thought that the gapmer binds to the target nucleic acid, at which point an RNAse is recruited and can then cleave the target nucleic acid. In some embodiments, the Y region is flanked both 5′ and 3′ by regions X and Z comprising high-affinity modified nucleotides, e.g., 1-6 modified nucleotides. Exemplary modified oligonucleotides include, but are not limited to, 2′ MOE or 2′OMe or Locked Nucleic Acid bases (LNA). The flanks X and Z may be have a of length 1-20 nucleotides, preferably 1-8 nucleotides and even more preferred 1-5 nucleotides. The flanks X and Z may be of similar length or of dissimilar lengths. The gap-segment Y may be a nucleotide sequence of length 5-20 nucleotides, preferably 6-12 nucleotides and even more preferred 6-10 nucleotides. In some aspects, the gap region of the gapmer oligonucleotides of the invention may contain modified nucleotides known to be acceptable for efficient RNase H action in addition to DNA nucleotides, such as C4′-substituted nucleotides, acyclic nucleotides, and arabino-configured nucleotides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using any method known in the art or described herein. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,432,250; and 7,683,036; U.S. patent publication Nos. US20090286969, US20100197762, and US20110112170; and PCT publication Nos. WO2008049085 and WO2009090182, each of which is herein incorporated by reference in its entirety.

### Formulation, Delivery, And Dosing

The oligonucleotides described herein can be formulated for administration to a subject for treating a condition (e.g., Friedrich's ataxia) associated with decreased levels of FXN. It should be understood that the formulations, compositions and methods can be practiced with any of the oligonucleotides disclosed herein.

The formulations may conveniently be presented in unit dosage form and may be prepared by any methods well known in the art of pharmacy. The amount of active ingredient (e.g., an oligonucleotide or compound of the invention) which can be combined with a carrier material to produce a single dosage form will vary depending upon the host being treated, the particular mode of administration, e.g., intrathecal, intraneural, intracerebral, intramuscular, etc. The amount of active ingredient which can be combined with a carrier material to produce a single dosage form will generally be that amount of the compound which produces a therapeutic effect.

Pharmaceutical formulations of this invention can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated oligonucleotide composition can assume a variety of states. In some examples, the composition is at least partially crystalline, uniformly crystalline, and/or anhydrous (e.g., less than 80, 50, 30, 20, or 10% water). In another example, the oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A oligonucleotide preparation can be formulated or administered (together or separately) in combination with another agent, e.g., another therapeutic agent or an agent that stabilizes an oligonucleotide, e.g., a protein that complexes with the oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, the oligonucleotide preparation includes another oligonucleotide, e.g., a second oligonucleotide that modulates expression of a second gene or a second oligonucleotide that modulates expression of the first gene. Still other preparation can include at least 3, 5, ten, twenty, fifty, or a hundred or more different oligonucleotide species. Such oligonucleotides can mediated gene expression with respect to a similar number of different genes. In one embodiment, the oligonucleotide preparation includes at least a second therapeutic agent (e.g., an agent other than an oligonucleotide).

### Route of Delivery

A composition that includes an oligonucleotide can be delivered to a subject by a variety of routes. Exemplary routes include: intrathecal, intraneural, intracerebral, intramuscular, oral, intravenous, intradermal, topical, rectal, parenteral, anal, intravaginal, intranasal, pulmonary, or ocular. The term “therapeutically effective amount” is the amount of oligonucleotide present in the composition that is needed to provide the desired level of FXN expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The oligonucleotide molecules of the invention can be incorporated into pharmaceutical compositions suitable for administration. Such compositions typically include one or more species of oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the oligonucleotide in aerosol form. The vascular endothelial cells could be targeted by coating a balloon catheter with the oligonucleotide and mechanically introducing the oligonucleotide. Targeting of neuronal cells could be accomplished by intrathecal, intraneural, intracerebral administration.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a composition disclosed herein via the transdermal route include hydration of the skin and the use of controlled release topical patches. The transdermal route provides a potentially effective means to deliver a composition disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, oligonucleotides administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the oligonucleotide can be applied, localized and removed easily.

In oral delivery, compositions can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A pharmaceutical composition of oligonucleotide may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the oligonucleotides described herein can be administered to ocular tissue. For example, the compositions can be applied to the surface of the eye or nearby tissue, e.g., the inside of the eyelid. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. The oligonucleotide can also be administered to the interior of the eye, and can be introduced by a needle or other delivery device which can introduce it to a selected area or structure.

Pulmonary delivery compositions can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. A oligonucleotide composition may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the composition has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide, e.g., a device can release insulin.

In one embodiment, unit doses or measured doses of a composition that includes oligonucleotide are dispensed by an implanted device. The device can include a sensor that monitors a parameter within a subject. For example, the device can include pump, e.g., and, optionally, associated electronics.

Tissue, e.g., cells or organs can be treated with an oligonucleotide, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, the oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

### Dosage

In one aspect, the invention features a method of administering an oligonucleotide (e.g., as a compound or as a component of a composition) to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with a reduced level of FXN. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of an oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some embodiments, the oligonucleotide pharmaceutical composition includes a plurality of oligonucleotide species. In another embodiment, the oligonucleotide species has sequences that are non-overlapping and non-adjacent to another species with respect to a target sequence (e.g., a region of a FXN target). In another embodiment, the plurality of oligonucleotide species is specific for different regions of a FXN target (such as different regions of a sequence as set forth in one of SEQ ID NOs: 110-113). In another embodiment, the oligonucleotide is allele specific.

In some cases, a patient is treated with an oligonucleotide in conjunction with other therapeutic modalities.

Following successful treatment, it may be desirable to have the patient undergo maintenance therapy to prevent the recurrence of the disease state, wherein the compound of the invention is administered in maintenance doses, ranging from 0.0001 mg to 100 mg per kg of body weight.

The concentration of the oligonucleotide composition is an amount sufficient to be effective in treating or preventing a disorder or to regulate a physiological condition in humans. The concentration or amount of oligonucleotide administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide can include a single treatment or, preferably, can include a series of treatments. It will also be appreciated that the effective dosage of an oligonucleotide used for treatment may increase or decrease over the course of a particular treatment. For example, the subject can be monitored after administering an oligonucleotide composition. Based on information from the monitoring, an additional amount of the oligonucleotide composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of FXN expression levels in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human FXN gene. In another embodiment, the composition for testing includes an oligonucleotide that is complementary, at least in an internal region, to a sequence that is conserved between a region of a FXN target in the animal model and a FXN target in a human.

In one embodiment, the administration of the oligonucleotide composition is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the invention, kits are provided, comprising a container housing a composition comprising an oligonucleotide. In some embodiments, the composition is a pharmaceutical composition comprising an oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present invention is further illustrated by the following Examples, which in no way should be construed as further limiting.

### EXAMPLES

### Example 1

### MATERIALS AND METHODS

### Real Time PCR

RNA analysis, cDNA synthesis and QRT-PCR were performed with Life Technologies Cells-to-Ct kit and StepOne Plus instrument. Baseline levels were also determined for mRNA of various housekeeping genes which are constitutively expressed. A “control” housekeeping gene with approximately the same level of baseline expression as the target gene was chosen for comparison purposes.

### ELISA

ELISA assays were performed as previously described using the Abcam Frataxin ELISA kit (ab115346).

### Cell Lines

Cells were cultured using conditions known in the art (see, e.g., Current Protocols in Cell Biology). Details of the cell lines used in the experiments described herein are provided in Table 1.

TABLE 1
Cell lines

| | Clinically | | # of GAA | |
|---|---|---|---|---|
| Cell lines | affected | Cell type | repeats | Notes |
| GM03816 | Y | Fibroblast | 330/380 | Coriell Cell Repository |
| GM0321B | N | Fibroblast | | Coriell Cell Repository |

Oligonucleotide Design

Oligonucleotides were designed to be complementary to a region of the sense strand of the human FXN gene and/or complementary to a region of the human FXN mRNA. The sequence and structure of each oligonucleotide is shown in Tables 2, 3, and 5. Table 4 provides a description of the nucleotide analogs, modifications and internucleoside linkages used for certain oligonucleotides described in Tables 2, 3, and 5. Certain oligos in Table 2 have two oligo names the “Oligo Name” and the “Alternative Oligo Name”, which are used interchangeably herein and are to be understood to refer to the same oligo.

TABLE 2
Oligonucleotides designed to target a region of FXN

| SEQ | | | | | | | |
|---|---|---|---|---|---|---|---|
| ID | Oligo | Alternative | Base | Targeting | Gene | | Formatted |
| NO | Name | Oligo Name | Sequence | Region | Name | Organism | Sequence |
| 1 | FXN-324 | Oligo324 | CGGCGCCCG | UTR/ | FXN | human | dCs; InaGs; dGs; InaCs; |
| | m02 | | AGAGTCCAC | internal | | | dGs; InaCs; dCs; InaCs; |
| | | | AT | | | | dGs; InaAs; dGs; InaAs; |
| | | | | | | | dGs; InaTs; dCs; |
| | | | | | | | InaCs; dAs; InaCs; |
| | | | | | | | dAs; InaT-Sup |
| 2 | FXN-325 | Oligo325 | CCAGGAGGC | Internal/ | FXN | human | dCs; InaCs; dAs; InaGs; |
| | m02 | | CGGCTACTG | exon | | | dGs; InaAs; dGs; InaGs; |
| | | | CG | | | | dCs; InaCs; dGs; |
| | | | | | | | InaGs; dCs; InaTs; dAs; |
| | | | | | | | InaCs; dTs; InaGs; |
| | | | | | | | dCs; InaG-Sup |
| 3 | FXN-326 | Oligo326 | CTGGGCTGG | Internal/ | FXN | human | dCs; InaTs; dGs; InaGs; |
| | m02 | | GCTGGGTGA | exon | | | dGs; InaCs; dTs; InaGs; |
| | | | CG | | | | dGs; InaGs; dCs; |
| | | | | | | | InaTs; dGs; InaGs; dGs; |
| | | | | | | | InaTs; dGs; InaAs; |
| | | | | | | | dCs; InaG-Sup |
| 4 | FXN-327 | Oligo327 | ACCCGGGTG | Internal/ | FXN | human | dAs; InaCs; dCs; InaCs; |
| | m02 | | AGGGTCTGG | exon | | | dGs; InaGs; dGs; InaTs; |
| | | | GC | | | | dGs; InaAs; dGs; |
| | | | | | | | InaGs; dGs; InaTs; dCs; |
| | | | | | | | InaTs; dGs; InaGs; |
| | | | | | | | dGs; InaC-Sup |
| 5 | FXN-328 | Oligo328 | CCAACTCTGC | Internal/ | FXN | human | dCs; InaCs; dAs; InaAs; |
| | m02 | | CGGCCGCGG | exon | | | dCs; InaTs; dCs; InaTs; |
| | | | G | | | | dGs; InaCs; dCs; |
| | | | | | | | InaGs; dGs; InaCs; dCs; |
| | | | | | | | InaGs; dCs; InaGs; dGs; |
| | | | | | | | InaG-Sup |
| 6 | FXN-329 | Oligo329 | ACGGCGGCC | Internal/ | FXN | human | dAs; InaCs; dGs; InaGs; |
| | m02 | | GCAGAGTGG | exon | | | dCs; InaGs; dGs; InaCs; |
| | | | GG | | | | dCs; InaGs; dCs; |
| | | | | | | | InaAs; dGs; InaAs; dGs; |
| | | | | | | | InaTs; dGs; InaGs; |
| | | | | | | | dGs; InaG-Sup |
| 7 | FXN-330 | Oligo330 | TCGATGTCG | Internal/ | FXN | human | dTs; InaCs; dGs; InaAs; |
| | m02 | | GTGCGCAGG | exon | | | dTs; InaGs; dTs; InaCs; |
| | | | CC | | | | dGs; InaGs; dTs; |
| | | | | | | | InaGs; dCs; InaGs; dCs; |
| | | | | | | | InaAs; dGs; InaGs; |
| | | | | | | | dCs; InaC-Sup |
| 8 | FXN-331 | Oligo331 | GGCGGGGCG | Internal/ | FXN | human | dGs; InaGs; dCs; InaGs; |
| | m02 | | TGCAGGTCG | exon | | | dGs; InaGs; dGs; InaCs; |
| | | | CA | | | | dGs; InaTs; dGs; |
| | | | | | | | InaCs; dAs; InaGs; dGs; |
| | | | | | | | InaTs; dCs; InaGs; |
| | | | | | | | dCs; InaA-Sup |
| 9 | FXN-332 | Oligo332 | ACGTTGGTTC | Internal/ | FXN | human | dAs; InaCs; dGs; InaTs; |
| | m02 | | GAACTTGCG | exon | | | dTs; InaGs; dGs; InaTs; |
| | | | C | | | | dTs; InaCs; dGs; InaAs; |
| | | | | | | | dAs; InaCs; dTs; |
| | | | | | | | InaTs; dGs; InaCs; dGs; |
| | | | | | | | InaC-Sup |
| 10 | FXN-333 | Oligo333 | TTCCAAATCT | Internal/ | FXN | human | dTs; InaTs; dCs; InaCs; |
| | m02 | | GGTTGAGGC | exon | | | dAs; InaAs; dAs; InaTs; |
| | | | C | | | | dCs; InaTs; dGs; InaGs; |
| | | | | | | | dTs; InaTs; dGs; |
| | | | | | | | InaAs; dGs; InaGs; dCs; |
| | | | | | | | InaC-Sup |
| 11 | FXN-334 | Oligo334 | AGACACTCTG | Internal/ | FXN | human | dAs; InaGs; dAs; InaCs; |
| | m02 | | CTTTTTGACA | exon | | | dAs; InaCs; dTs; InaCs; |
| | | | | | | | dTs; InaGs; dCs; InaTs; |
| | | | | | | | dTs; InaTs; dTs; |
| | | | | | | | InaTs; dGs; InaAs; dCs; |
| | | | | | | | InaA-Sup |
| 12 | FXN-335 | Oligo335 | TTTCCTCAAA | Internal/ | FXN | human | dTs; InaTs; dTs; InaCs; |
| | m02 | | TTCATCAAAT | exon | | | dCs; InaTs; dCs; InaAs; |
| | | | | | | | dAs; InaAs; dTs; InaTs; |
| | | | | | | | dCs; InaAs; dTs; InaCs; |
| | | | | | | | dAs; InaAs; dAs; |
| | | | | | | | InaT-Sup |
| 13 | FXN-336 | Oligo336 | GGGTGGCCC | Internal/ | FXN | human | dGs; InaGs; dGs; InaTs; |
| | m02 | | AAAGTTCCA | exon | | | dGs; InaGs; dCs; InaCs; |
| | | | GA | | | | dCs; InaAs; dAs; InaAs; |
| | | | | | | | dGs; InaTs; dTs; |
| | | | | | | | InaCs; dCs; InaAs; |
| | | | | | | | dGs; InaA-Sup |
| 14 | FXN-337 | Oligo337 | TGGTCTCATC | Internal/ | FXN | human | dTs; InaGs; dGs; InaTs; |
| | m02 | | TAGAGAGCC | exon | | | dCs; InaTs; dCs; InaAs; |
| | | | T | | | | dTs; InaCs; dTs; InaAs; |
| | | | | | | | dGs; InaAs; dGs; |
| | | | | | | | InaAs; dGs; InaCs; dCs; |
| | | | | | | | InaT-Sup |
| 15 | FXN-338 | Oligo338 | CTCTGCTAGT | Internal/ | FXN | human | dCs; InaTs; dCs; InaTs; |
| | m02 | | CTTTCATAGG | exon | | | dGs; InaCs; dTs; InaAs; |
| | | | | | | | dGs; InaTs; dCs; InaTs; |
| | | | | | | | dTs; InaTs; dCs; InaAs; |
| | | | | | | | dTs; InaAs; dGs; |
| | | | | | | | InaG-Sup |
| 16 | FXN-339 | Oligo339 | GCTAAAGAG | Internal/ | FXN | human | dGs; InaCs; dTs; InaAs; |
| | m02 | | TCCAGCGTTT | exon | | | dAs; InaAs; dGs; InaAs; |
| | | | C | | | | dGs; InaTs; dCs; InaCs; |
| | | | | | | | dAs; InaGs; dCs; |
| | | | | | | | InaGs; dTs; InaTs; dTs; |
| | | | | | | | InaC-Sup |
| 17 | FXN-340 | Oligo340 | GCAAGGTCT | Internal/ | FXN | human | dGs; InaCs; dAs; InaAs; |
| | m02 | | TCAAAAAACT | exon | | | dGs; InaGs; dTs; InaCs; |
| | | | CT | | | | dTs; InaTs; dCs; InaAs; |
| | | | | | | | dAs; InaAs; dAs; |
| | | | | | | | InaAs; dAs; InaCs; dTs; |
| | | | | | | | InaCs; dT-Sup |
| 18 | FXN-341 | Oligo341 | CTCAAACGTG | Internal/ | FXN | human | dCs; InaTs; dCs; InaAs; |
| | m02 | | TATGGCTTGT | exon | | | dAs; InaAs; dCs; InaGs; |
| | | | CT | | | | dTs; InaGs; dTs; InaAs; |
| | | | | | | | dTs; InaGs; dGs; |
| | | | | | | | InaCs; dTs; InaTs; dGs; |
| | | | | | | | InaTs; dCs; InaT- |
| | | | | | | | Sup |
| 19 | FXN-342 | Oligo342 | CCCAAAGGA | Internal/ | FXN | human | dCs; InaCs; dCs; InaAs; |
| | m02 | | GACATCATA | exon | | | dAs; InaAs; dGs; InaGs; |
| | | | GTC | | | | dAs; InaGs; dAs; |
| | | | | | | | InaCs; dAs; InaTs; dCs; |
| | | | | | | | InaAs; dTs; InaAs; |
| | | | | | | | dGs; InaTs; dC-Sup |
| 20 | FXN-343 | Oligo343 | CAGTTTGACA | Internal/ | FXN | human | dCs; InaAs; dGs; InaTs; |
| | m02 | | GTTAAGACA | exon | | | dTs; InaTs; dGs; InaAs; |
| | | | CCACT | | | | dCs; InaAs; dGs; |
| | | | | | | | InaTs; dTs; InaAs; dAs; |
| | | | | | | | InaGs; dAs; InaCs; |
| | | | | | | | dAs; InaCs; dCs; InaAs; |
| | | | | | | | dCs; InaT-Sup |
| 21 | FXN-344 | Oligo344 | ATAGGTTCCT | Internal/ | FXN | human | dAs; InaTs; dAs; InaGs; |
| | m02 | | AGATCTCCAC | exon | | | dGs; InaTs; dTs; InaCs; |
| | | | C | | | | dCs; InaTs; dAs; InaGs; |
| | | | | | | | dAs; InaTs; dCs; |
| | | | | | | | InaTs; dCs; InaCs; dAs; |
| | | | | | | | InaCs; dC-Sup |
| 22 | FXN-345 | Oligo345 | GGCGTCTGC | Internal/ | FXN | human | dGs; InaGs; dCs; InaGs; |
| | m02 | | TTGTTGATCA | exon | | | dTs; InaCs; dTs; InaGs; |
| | | | C | | | | dCs; InaTs; dTs; InaGs; |
| | | | | | | | dTs; InaTs; dGs; |
| | | | | | | | InaAs; dTs; InaCs; dAs; |
| | | | | | | | InaC-Sup |
| 23 | FXN-346 | Oligo346 | AAGATAGCC | Internal/ | FXN | human | dAs; InaAs; dGs; InaAs; |
| | m02 | | AGATTTGCTT | exon | | | dTs; InaAs; dGs; InaCs; |
| | | | GTTT | | | | dCs; InaAs; dGs; InaAs; |
| | | | | | | | dTs; InaTs; dTs; |
| | | | | | | | InaGs; dCs; InaTs; dTs; |
| | | | | | | | InaGs; dTs; InaTs; |
| | | | | | | | dT-Sup |
| 24 | FXN-347 | Oligo347 | GGTCCACTAC | Internal/ | FXN | human | dGs; InaGs; dTs; InaCs; |
| | m02 | | ATACCTGGAT | exon | | | dCs; InaAs; dCs; InaTs; |
| | | | GGAG | | | | dAs; InaCs; dAs; InaTs; |
| | | | | | | | dAs; InaCs; dCs; |
| | | | | | | | InaTs; dGs; InaGs; dAs; |
| | | | | | | | InaTs; dGs; InaGs; |
| | | | | | | | dAs; InaG-Sup |
| 25 | FXN-348 | Oligo348 | CCCAGTCCAG | Internal/ | FXN | human | dCs; InaCs; dCs; InaAs; |
| | m02 | | TCATAACGCT | exon | | | dGs; InaTs; dCs; InaCs; |
| | | | T | | | | dAs; InaGs; dTs; InaCs; |
| | | | | | | | dAs; InaTs; dAs; |
| | | | | | | | InaAs; dCs; InaGs; dCs; |
| | | | | | | | InaTs; dT-Sup |
| 26 | FXN-349 | Oligo349 | CGTGGGAGT | Internal/ | FXN | human | dCs; InaGs; dTs; InaGs; |
| | m02 | | ACACCCAGTT | exon | | | dGs; InaGs; dAs; InaGs; |
| | | | TTT | | | | dTs; InaAs; dCs; InaAs; |
| | | | | | | | dCs; InaCs; dCs; |
| | | | | | | | InaAs; dGs; InaTs; |
| | | | | | | | dTs; InaTs; dTs; InaT- |
| | | | | | | | Sup |
| 27 | FXN-350 | Oligo350 | CATGGAGGG | Internal/ | FXN | human | dCs; InaAs; dTs; InaGs; |
| | m02 | | ACACGCCGT | exon | | | dGs; InaAs; dGs; InaGs; |
| | | | | | | | dGs; InaAs; dCs; |
| | | | | | | | InaAs; dCs; InaGs; dCs; |
| | | | | | | | InaCs; dGs; InaT- |
| | | | | | | | Sup |
| 28 | FXN-351 | Oligo351 | GTGAGCTCT | Internal/ | FXN | human | dGs; InaTs; dGs; InaAs; |
| | m02 | | GCGGCCAGC | exon | | | dGs; InaCs; dTs; InaCs; |
| | | | AGCT | | | | dTs; InaGs; dCs; InaGs; |
| | | | | | | | dGs; InaCs; dCs; |
| | | | | | | | InaAs; dGs; InaCs; dAs; |
| | | | | | | | InaGs; dCs; InaT- |
| | | | | | | | Sup |
| 29 | FXN-352 | Oligo352 | AGTTTGGTTT | Internal/ | FXN | human | dAs; InaGs; dTs; InaTs; |
| | m02 | | TTAAGGCTTT | exon | | | dTs; InaGs; dGs; InaTs; |
| | | | A | | | | dTs; InaTs; dTs; InaTs; |
| | | | | | | | dAs; InaAs; dGs; |
| | | | | | | | InaGs; dCs; InaTs; dTs; |
| | | | | | | | InaTs; dA-Sup |
| 30 | FXN-353 | Oligo353 | TAGGCCAAG | Internal/ | FXN | human | dTs; InaAs; dGs; InaGs; |
| | m02 | | GAAGACAAG | exon | | | dCs; InaCs; dAs; InaAs; |
| | | | TCC | | | | dGs; InaGs; dAs; |
| | | | | | | | InaAs; dGs; InaAs; dCs; |
| | | | | | | | InaAs; dAs; InaGs; |
| | | | | | | | dTs; InaCs; dC-Sup |
| 31 | FXN-354 | Oligo354 | TCAAGCATCT | Internal/ | FXN | human | dTs; InaCs; dAs; InaAs; |
| | m02 | | TTTCCGGAA | exon | | | dGs; InaCs; dAs; InaTs; |
| | | | | | | | dCs; InaTs; dTs; InaTs; |
| | | | | | | | dTs; InaCs; dCs; |
| | | | | | | | InaGs; dGs; InaAs; dA- |
| | | | | | | | Sup |
| 32 | FXN-355 | Oligo355 | TCCTTAAAAC | UTR | FXN | human | dTs; InaCs; dCs; InaTs; |
| | m02 | | GGGGCTGGG | | | | dTs; InaAs; dAs; InaAs; |
| | | | CA | | | | dAs; InaCs; dGs; |
| | | | | | | | InaGs; dGs; InaGs; dCs; |
| | | | | | | | InaTs; dGs; InaGs; |
| | | | | | | | dGs; InaCs; dA-Sup |
| 33 | FXN-356 | Oligo356 | TTGGCCTGAT | UTR | FXN | human | dTs; InaTs; dGs; InaGs; |
| | m02 | | AGCTTTTAAT | | | | dCs; InaCs; dTs; InaGs; |
| | | | G | | | | dAs; InaTs; dAs; |
| | | | | | | | InaGs; dCs; InaTs; dTs; |
| | | | | | | | InaTs; dTs; InaAs; dAs; |
| | | | | | | | InaTs; dG-Sup |
| 34 | FXN-357 | Oligo357 | CCTCAGCTGC | UTR | FXN | human | dCs; InaCs; dTs; InaCs; |
| | m02 | | ATAATGAAG | | | | dAs; InaGs; dCs; InaTs; |
| | | | CTGGGGTC | | | | dGs; InaCs; dAs; InaTs; |
| | | | | | | | dAs; InaAs; dTs; |
| | | | | | | | InaGs; dAs; InaAs; dGs; |
| | | | | | | | InaCs; dTs; InaGs; |
| | | | | | | | dGs; InaGs; dGs; InaTs; |
| | | | | | | | dC-Sup |
| 35 | FXN-358 | Oligo358 | AACAACAAC | UTR | FXN | human | dAs; InaAs; dCs; InaAs; |
| | m02 | | AACAACAAA | | | | dAs; InaCs; dAs; InaAs; |
| | | | AAACAGA | | | | dCs; InaAs; dAs; |
| | | | | | | | InaCs; dAs; InaAs; dCs; |
| | | | | | | | InaAs; dAs; InaAs; |
| | | | | | | | dAs; InaAs; dAs; InaCs; |
| | | | | | | | dAs; InaGs; dA-Sup |
| 36 | FXN-359 | Oligo359 | CCTCAAAAGC | UTR | FXN | human | dCs; InaCs; dTs; InaCs; |
| | m02 | | AGGAATAAA | | | | dAs; InaAs; dAs; InaAs; |
| | | | AAAAATA | | | | dGs; InaCs; dAs; |
| | | | | | | | InaGs; dGs; InaAs; dAs; |
| | | | | | | | InaTs; dAs; InaAs; |
| | | | | | | | dAs; InaAs; dAs; InaAs; |
| | | | | | | | dAs; InaAs; dTs; InaA- |
| | | | | | | | Sup |
| 37 | FXN-360 | Oligo360 | GCTGTGACA | UTR | FXN | human | dGs; InaCs; dTs; InaGs; |
| | m02 | | CATAGCCCAA | | | | dTs; InaGs; dAs; InaCs; |
| | | | CTGT | | | | dAs; InaCs; dAs; InaTs; |
| | | | | | | | dAs; InaGs; dCs; InaCs; |
| | | | | | | | dCs; InaAs; dAs; |
| | | | | | | | InaCs; dTs; InaGs; dT- |
| | | | | | | | Sup |
| 38 | FXN-361 | Oligo361 | GGAGGCAAC | UTR | FXN | human | dGs; InaGs; dAs; InaGs; |
| | m02 | | ACATTCTTTC | | | | dGs; InaCs; dAs; |
| | | | TACAGA | | | | InaAs; dCs; InaAs; dCs; |
| | | | | | | | InaAs; dTs; InaTs; dCs; |
| | | | | | | | InaTs; dTs; InaTs; |
| | | | | | | | dCs; InaTs; dAs; InaCs; |
| | | | | | | | dAs; InaGs; dA-Sup |
| 39 | FXN-362 | Oligo362 | CTATTAATAT | intron | FXN | human | dCs; InaTs; dAs; InaTs; |
| | m02 | | TACTG | | | | dTs; InaAs; dAs; InaTs; |
| | | | | | | | dAs; InaTs; dTs; InaAs; |
| | | | | | | | dCs; InaTs; dG- |
| | | | | | | | Sup |
| 40 | FXN-363 | Oligo363 | CATTATGTGT | intron | FXN | human | dCs; InaAs; dTs; InaTs; |
| | m02 | | ATGTAT | | | | dAs; InaTs; dGs; InaTs; |
| | | | | | | | dGs; InaTs; dAs; InaTs; |
| | | | | | | | dGs; InaTs; dAs; |
| | | | | | | | InaT-Sup |
| 41 | FXN-364 | Oligo364 | TTTATCTATG | intron | FXN | human | dTs; InaTs; dTs; InaAs; |
| | m02 | | TTATT | | | | dTs; InaCs; dTs; InaAs; |
| | | | | | | | dTs; InaGs; dTs; InaTs; |
| | | | | | | | dAs; InaTs; dT- |
| | | | | | | | Sup |
| 42 | FXN-365 | Oligo365 | CTAATTTGAA | intron | FXN | human | dCs; InaTs; dAs; InaAs; |
| | m02 | | GTTCT | | | | dTs; InaTs; dTs; InaGs; |
| | | | | | | | dAs; InaAs; dGs; InaTs; |
| | | | | | | | dTs; InaCs; dT- |
| | | | | | | | Sup |
| 43 | FXN-366 | Oligo366 | TTCGAACTTG | Exon | FXN | human | dTs; InaTs; dCs; InaGs; |
| | m02 | | CGCGG | | | | dAs; InaAs; dCs; InaTs; |
| | | | | | | | dTs; InaGs; dCs; InaGs; |
| | | | | | | | dCs; InaGs; dG- |
| | | | | | | | Sup |
| 44 | FXN-367 | Oligo367 | TAGAGAGCC | Exon | FXN | human | dTs; InaAs; dGs; InaAs; |
| | m02 | | TGGGT | | | | dGs; InaAs; dGs; InaCs; |
| | | | | | | | dCs; InaTs; dGs; |
| | | | | | | | InaGs; dGs; InaT-Sup |
| 45 | FXN-368 | Oligo368 | ACACCACTCC | Exon | FXN | human | dAs; InaCs; dAs; InaCs; |
| | m02 | | CAAAG | | | | dCs; InaAs; dCs; InaTs; |
| | | | | | | | dCs; InaCs; dCs; InaAs; |
| | | | | | | | dAs; InaAs; dG- |
| | | | | | | | Sup |
| 46 | FXN-369 | Oligo369 | AGGTCCACTA | Exon | FXN | human | dAs; InaGs; dGs; InaTs; |
| | m02 | | CATAC | | | | dCs; InaCs; dAs; InaCs; |
| | | | | | | | dTs; InaAs; dCs; |
| | | | | | | | InaAs; dTs; InaAs; dC- |
| | | | | | | | Sup |
| 47 | FXN-370 | Oligo370 | CGTTAACCTG | Exon | FXN | human | dCs; InaGs; dTs; InaTs; |
| | m02 | | GATGG | | | | dAs; InaAs; dCs; InaCs; |
| | | | | | | | dTs; InaGs; dGs; |
| | | | | | | | InaAs; dTs; InaGs; dG- |
| | | | | | | | Sup |
| 48 | FXN-371 | Oligo371 | TGACCCAAG | | FXN | human | dTs; InaGs; dAs; InaCs; |
| | m02 | | GGAGAC | | | | dCs; InaCs; dAs; InaAs; |
| | | | | | | | dGs; InaGs; dGs; |
| | | | | | | | InaAs; dGs; InaAs; dC- |
| | | | | | | | Sup |
| 49 | FXN-372 | Oligo372 | TGGCCACTG | | FXN | human | dTs; InaGs; dGs; InaCs; |
| | m02 | | GCCGCA | | | | dCs; InaAs; dCs; InaTs; |
| | | | | | | | dGs; InaGs; dCs; |
| | | | | | | | InaCs; dGs; InaCs; dA- |
| | | | | | | | Sup |
| 50 | FXN-373 | Oligo373 | CGGCGACCC | | FXN | human | dCs; InaGs; dGs; InaCs; |
| | m02 | | CTGGTG | | | | dGs; InaAs; dCs; InaCs; |
| | | | | | | | dCs; InaCs; dTs; |
| | | | | | | | InaGs; dGs; InaTs; dG- |
| | | | | | | | Sup |
| 51 | FXN-374 | Oligo374 | CGCCCTCCAG | | FXN | human | dCs; InaGs; dCs; InaCs; |
| | m02 | | CGCTG | | | | dCs; InaTs; dCs; InaCs; |
| | | | | | | | dAs; InaGs; dCs; |
| | | | | | | | InaGs; dCs; InaTs; dG- |
| | | | | | | | Sup |
| 52 | FXN-375 | Oligo375 | CGCTCCGCCC | | FXN | human | dCs; InaGs; dCs; InaTs; |
| | m02 | | TCCAG | | | | dCs; InaCs; dGs; InaCs; |
| | | | | | | | dCs; InaCs; dTs; |
| | | | | | | | InaCs; dCs; InaAs; dG- |
| | | | | | | | Sup |
| 53 | FXN-376 | Oligo376 | TGACCCAAG | | FXN | human | dTs; InaGs; dAs; InaCs; |
| | m02 | | GGAGACCC | | | | dCs; InaCs; dAs; InaAs; |
| | | | | | | | dGs; InaGs; dGs; |
| | | | | | | | InaAs; dGs; InaAs; dCs; |
| | | | | | | | InaCs; dC-Sup |
| 54 | FXN-377 | Oligo377 | TGGCCACTG | | FXN | human | dTs; InaGs; dGs; InaCs; |
| | m02 | | GCCGCACC | | | | dCs; InaAs; dCs; InaTs; |
| | | | | | | | dGs; InaGs; dCs; |
| | | | | | | | InaCs; dGs; InaCs; dAs; |
| | | | | | | | InaCs; dC-Sup |
| 55 | FXN-378 | Oligo378 | CGGCGACCC | | FXN | human | dCs; InaGs; dGs; InaCs; |
| | m02 | | CTGGTGCC | | | | dGs; InaAs; dCs; InaCs; |
| | | | | | | | dCs; InaCs; dTs; |
| | | | | | | | InaGs; dGs; InaTs; dGs; |
| | | | | | | | InaCs; dC-Sup |
| 56 | FXN-379 | Oligo379 | CGCCCTCCAG | | FXN | human | dCs; InaGs; dCs; InaCs; |
| | m02 | | CGCTGCC | | | | dCs; InaTs; dCs; InaCs; |
| | | | | | | | dAs; InaGs; dCs; |
| | | | | | | | InaGs; dCs; InaTs; dGs; |
| | | | | | | | InaCs; dC-Sup |
| 57 | FXN-380 | Oligo380 | CGCTCCGCCC | | FXN | human | dCs; InaGs; dCs; InaTs; |
| | m02 | | TCCAGCC | | | | dCs; InaCs; dGs; InaCs; |
| | | | | | | | dCs; InaCs; dTs; |
| | | | | | | | InaCs; dCs; InaAs; dGs; |
| | | | | | | | InaCs; dC-Sup |
| 58 | FXN-381 | Oligo381 | TGACCCAAG | | FXN | human | dTs; InaGs; dAs; InaCs; |
| | m1000 | | GGAGACGGA | | | | dCs; InaCs; dAs; InaAs; |
| | | | AACCAC | | | | dGs; InaGs; dGs; |
| | | | | | | | InaAs; dGs; InaAs; dCs; |
| | | | | | | | InaGs; dGs; dAs; dAs; |
| | | | | | | | dAs; dCs; InaCs; dAs; |
| | | | | | | | InaC-Sup |
| 59 | FXN-382 | Oligo382 | TGGCCACTG | | FXN | human | dTs; InaGs; dGs; InaCs; |
| | m1000 | | GCCGCAGGA | | | | dCs; InaAs; dCs; InaTs; |
| | | | AACCAC | | | | dGs; InaGs; dCs; |
| | | | | | | | InaCs; dGs; InaCs; dAs; |
| | | | | | | | InaGs; dGs; dAs; dAs; |
| | | | | | | | dAs; dCs; InaCs; dAs; |
| | | | | | | | InaC-Sup |
| 60 | FXN-383 | Oligo383 | CGGCGACCC | | FXN | human | dCs; InaGs; dGs; InaCs; |
| | m1000 | | CTGGTGGGA | | | | dGs; InaAs; dCs; InaCs; |
| | | | AACCTC | | | | dCs; InaCs; dTs; |
| | | | | | | | InaGs; dGs; InaTs; dGs; |
| | | | | | | | InaGs; dGs; dAs; dAs; |
| | | | | | | | dAs; dCs; InaCs; dTs; |
| | | | | | | | InaC-Sup |
| 61 | FXN-384 | Oligo384 | CGCCCTCCAG | | FXN | human | dCs; InaGs; dCs; InaCs; |
| | m1000 | | CGCTGGGAA | | | | dCs; InaTs; dCs; InaCs; |
| | | | ACCTC | | | | dAs; InaGs; dCs; |
| | | | | | | | InaGs; dCs; InaTs; dGs; |
| | | | | | | | InaGs; dGs; dAs; dAs; |
| | | | | | | | dAs; dCs; InaCs; dTs; |
| | | | | | | | InaC-Sup |
| 62 | FXN-385 | Oligo385 | CGCTCCGCCC | | FXN | human | dCs; InaGs; dCs; InaTs; |
| | m1000 | | TCCAGCCAAA | | | | dCs; InaCs; dGs; InaCs; |
| | | | GGTC | | | | dCs; InaCs; dTs; InaCs; |
| | | | | | | | dCs; InaAs; dGs; |
| | | | | | | | InaCs; dCs; dAs; dAs; |
| | | | | | | | dAs; dGs; InaGs; dTs; |
| | | | | | | | InaC-Sup |
| 63 | FXN-386 | Oligo386 | GGTTTTTAAG | | FXN | human | dGs; InaGs; dTs; InaTs; |
| | m02 | | GCTTT | | | | dTs; InaTs; dTs; InaAs; |
| | | | | | | | dAs; InaGs; dGs; InaCs; |
| | | | | | | | dTs; InaTs; dT- |
| | | | | | | | Sup |
| 64 | FXN-387 | Oligo387 | GGGGTCTTG | | FXN | human | dGs; InaGs; dGs; InaGs; |
| | m02 | | GCCTGA | | | | dTs; InaCs; dTs; InaTs; |
| | | | | | | | dGs; InaGs; dCs; |
| | | | | | | | InaCs; dTs; InaGs; dA- |
| | | | | | | | Sup |
| 65 | FXN-388 | Oligo388 | CATAATGAA | | FXN | human | dCs; InaAs; dTs; InaAs; |
| | m02 | | GCTGGG | | | | dAs; InaTs; dGs; InaAs; |
| | | | | | | | dAs; InaGs; dCs; |
| | | | | | | | InaTs; dGs; InaGs; dG- |
| | | | | | | | Sup |
| 66 | FXN-389 | Oligo389 | AGGAGGCAA | | FXN | human | dAs; InaGs; dGs; InaAs; |
| | m02 | | CACATT | | | | dGs; InaGs; dCs; InaAs; |
| | | | | | | | dAs; InaCs; dAs; |
| | | | | | | | InaCs; dAs; InaTs; dT- |
| | | | | | | | Sup |
| 67 | FXN-390 | Oligo390 | ATTATTTTGC | | FXN | human | dAs; InaTs; dTs; InaAs; |
| | m02 | | TTTTT | | | | dTs; InaTs; dTs; InaTs; |
| | | | | | | | dGs; InaCs; dTs; |
| | | | | | | | InaTs; dTs; InaTs; dT- |
| | | | | | | | Sup |
| 68 | FXN-391 | Oligo391 | CATTTTCCCT | | FXN | human | dCs; InaAs; dTs; InaTs; |
| | m02 | | CCTGG | | | | dTs; InaTs; dCs; InaCs; |
| | | | | | | | dCs; InaTs; dCs; |
| | | | | | | | InaCs; dTs; InaGs; dG- |
| | | | | | | | Sup |
| 69 | FXN-392 | Oligo392 | GTAGGCTAC | | FXN | human | dGs; InaTs; dAs; InaGs; |
| | m02 | | CCTTTA | | | | dGs; InaCs; dTs; InaAs; |
| | | | | | | | dCs; InaCs; dCs; InaTs; |
| | | | | | | | dTs; InaTs; dA- |
| | | | | | | | Sup |
| 70 | FXN-393 | Oligo393 | GAGGCTTGT | | FXN | human | dGs; InaAs; dGs; InaGs; |
| | m02 | | TGCTTT | | | | dCs; InaTs; dTs; InaGs; |
| | | | | | | | dTs; InaTs; dGs; |
| | | | | | | | InaCs; dTs; InaTs; dT- |
| | | | | | | | Sup |
| 71 | FXN-394 | Oligo394 | CATGTATGAT | | FXN | human | dCs; InaAs; dTs; InaGs; |
| | m02 | | GTTAT | | | | dTs; InaAs; dTs; InaGs; |
| | | | | | | | dAs; InaTs; dGs; |
| | | | | | | | InaTs; dTs; InaAs; dT- |
| | | | | | | | Sup |
| 72 | FXN-395 | Oligo395 | TTTTTGGTTT | | FXN | human | dTs; InaTs; dTs; InaTs; |
| | m02 | | TTAAGGCTTT | | | | dTs; InaGs; dGs; InaTs; |
| | | | | | | | dTs; InaTs; dTs; InaTs; |
| | | | | | | | dAs; InaAs; dGs; |
| | | | | | | | InaGs; dCs; InaTs; dTs; |
| | | | | | | | InaT-Sup |
| 73 | FXN-396 | Oligo396 | TTTTTGGGGT | | FXN | human | dTs; InaTs; dTs; InaTs; |
| | m02 | | CTTGGCCTGA | | | | dTs; InaGs; dGs; InaGs; |
| | | | | | | | dGs; InaTs; dCs; |
| | | | | | | | InaTs; dTs; InaGs; dGs; |
| | | | | | | | InaCs; dCs; InaTs; dGs; |
| | | | | | | | InaA-Sup |
| 74 | FXN-397 | Oligo397 | TTTTTCATAA | | FXN | human | dTs; InaTs; dTs; InaTs; |
| | m02 | | TGAAGCTGG | | | | dTs; InaCs; dAs; InaTs; |
| | | | G | | | | dAs; InaAs; dTs; |
| | | | | | | | InaGs; dAs; InaAs; dGs; |
| | | | | | | | InaCs; dTs; InaGs; |
| | | | | | | | dGs; InaG-Sup |
| 75 | FXN-398 | Oligo398 | TTTTTAGGAG | | FXN | human | dTs; InaTs; dTs; InaTs; |
| | m02 | | GCAACACATT | | | | dTs; InaAs; dGs; InaGs; |
| | | | | | | | dAs; InaGs; dGs; |
| | | | | | | | InaCs; dAs; InaAs; dCs; |
| | | | | | | | InaAs; dCs; InaAs; |
| | | | | | | | dTs; InaT-Sup |
| 76 | FXN-399 | Oligo399 | TTTTTATTATT | | FXN | human | dTs; InaTs; dTs; InaTs; |
| | m02 | | TTGCTTTTT | | | | dTs; InaAs; dTs; InaTs; |
| | | | | | | | dAs; InaTs; dTs; |
| | | | | | | | InaTs; dTs; InaGs; dCs; |
| | | | | | | | InaTs; dTs; InaTs; dTs; |
| | | | | | | | InaT-Sup |
| 77 | FXN-429 | Oligo429 | ATGGGGGAC | | FXN | human | InaAs; InaTs; InaGs; |
| | m02 | | GGGGCA | | | | dGs; dGs; dGs; dGs; dAs; |
| | | | | | | | dCs; dGs; dGs; dGs; |
| | | | | | | | InaGs; InaCs; InaA- |
| | | | | | | | Sup |
| 78 | FXN-415 | Oligo415 | GGTTGAGAC | | FXN | human | dGs; InaGs; dTs; InaTs; |
| | m02 | | TGGGTG | | | | dGs; InaAs; dGs; |
| | | | | | | | InaAs; dCs; InaTs; dGs; |
| | | | | | | | InaGs; dGs; InaTs; dG- |
| | | | | | | | Sup |
| 79 | FXN-414 | Oligo414 | ATGGGGGAC | | FXN | human | dAs; InaTs; dGs; InaGs; |
| | m02 | | GGGGCA | | | | dGs; InaGs; dGs; InaAs; |
| | | | | | | | dCs; InaGs; dGs; |
| | | | | | | | InaGs; dGs; InaCs; dA- |
| | | | | | | | Sup |

TABLE 3
Oligonucleotides designed to target a region of FXN

| SEQ | | | | | | |
|---|---|---|---|---|---|---|
| ID | | Base | Targeting | Gene | | |
| NO | Oligo Name | Sequence | Region | Name | Organism | Formatted Sequence |
| 1 | Oligo1 | CGGCGCCCG | Internal | FXN | human | dCs; InaGs; dGs; InaCs; dGs; |
| | | AGAGTCCAC | | | | InaCs; dCs; InaCs; dGs; InaAs; |
| | | AT | | | | dGs; InaAs; dGs; InaTs; dCs; |
| | | | | | | InaCs; dAs; InaCs; dAs; InaT-Sup |
| 2 | Oligo2 | CCAGGAGGC | Internal | FXN | human | dCs; InaCs; dAs; InaGs; dGs; |
| | | CGGCTACTG | | | | InaAs; dGs; InaGs; dCs; InaCs; |
| | | CG | | | | dGs; InaGs; dCs; InaTs; dAs; |
| | | | | | | InaCs; dTs; InaGs; dCs; InaG-Sup |
| 3 | Oligo3 | CTGGGCTGG | Internal | FXN | human | dCs; InaTs; dGs; InaGs; dGs; |
| | | GCTGGGTGA | | | | InaCs; dTs; InaGs; dGs; InaGs; |
| | | CG | | | | dCs; InaTs; dGs; InaGs; dGs; |
| | | | | | | InaTs; dGs; InaAs; dCs; InaG-Sup |
| 4 | Oligo4 | ACCCGGGTG | Internal | FXN | human | dAs; InaCs; dCs; InaCs; dGs; |
| | | AGGGTCTGG | | | | InaGs; dGs; InaTs; dGs; InaAs; |
| | | GC | | | | dGs; InaGs; dGs; InaTs; dCs; |
| | | | | | | InaTs; dGs; InaGs; dGs; InaC-Sup |
| 5 | Oligo5 | CCAACTCTG | Internal | FXN | human | dCs; InaCs; dAs; InaAs; dCs; |
| | | CCGGCCGCG | | | | InaTs; dCs; InaTs; dGs; InaCs; |
| | | GG | | | | dCs; InaGs; dGs; InaCs; dCs; |
| | | | | | | InaGs; dCs; InaGs; dGs; InaG-Sup |
| 6 | Oligo6 | ACGGCGGCC | Internal | FXN | human | dAs; InaCs; dGs; InaGs; dCs; |
| | | GCAGAGTGG | | | | InaGs; dGs; InaCs; dCs; InaGs; |
| | | GG | | | | dCs; InaAs; dGs; InaAs; dGs; |
| | | | | | | InaTs; dGs; InaGs; dGs; InaG-Sup |
| 7 | Oligo7 | TCGATGTCG | Internal | FXN | human | dTs; InaCs; dGs; InaAs; dTs; |
| | | GTGCGCAGG | | | | InaGs; dTs; InaCs; dGs; InaGs; |
| | | CC | | | | dTs; InaGs; dCs; InaGs; dCs; InaAs; |
| | | | | | | dGs; InaGs; dCs; InaC-Sup |
| 8 | Oligo8 | GGCGGGGC | Internal | FXN | human | dGs; InaGs; dCs; InaGs; dGs; |
| | | GTGCAGGTC | | | | InaGs; dGs; InaCs; dGs; InaTs; |
| | | GCA | | | | dGs; InaCs; dAs; InaGs; dGs; |
| | | | | | | InaTs; dCs; InaGs; dCs; InaA-Sup |
| 9 | Oligo9 | ACGTTGGTT | Internal | FXN | human | dAs; InaCs; dGs; InaTs; dTs; |
| | | CGAACTTGC | | | | InaGs; dGs; InaTs; dTs; InaCs; dGs; |
| | | GC | | | | InaAs; dAs; InaCs; dTs; InaTs; |
| | | | | | | dGs; InaCs; dGs; InaC-Sup |
| 10 | Oligo10 | TTCCAAATCT | Internal | FXN | human | dTs; InaTs; dCs; InaCs; dAs; InaAs; |
| | | GGTTGAGGC | | | | dAs; InaTs; dCs; InaTs; dGs; |
| | | C | | | | InaGs; dTs; InaTs; dGs; InaAs; |
| | | | | | | dGs; InaGs; dCs; InaC-Sup |
| 11 | Oligo11 | AGACACTCT | Internal | FXN | human | dAs; InaGs; dAs; InaCs; dAs; InaCs; |
| | | GCTTTTTGAC | | | | dTs; InaCs; dTs; InaGs; dCs; |
| | | A | | | | InaTs; dTs; InaTs; dTs; InaTs; |
| | | | | | | dGs; InaAs; dCs; InaA-Sup |
| 12 | Oligo12 | TTTCCTCAAA | Internal | FXN | human | dTs; InaTs; dTs; InaCs; dCs; InaTs; |
| | | TTCATCAAAT | | | | dCs; InaAs; dAs; InaAs; dTs; |
| | | | | | | InaTs; dCs; InaAs; dTs; InaCs; |
| | | | | | | dAs; InaAs; dAs; InaT-Sup |
| 13 | Oligol3 | GGGTGGCCC | Internal | FXN | human | dGs; InaGs; dGs; InaTs; dGs; InaGs; |
| | | AAAGTTCCA | | | | dCs; InaCs; dCs; InaAs; dAs; |
| | | GA | | | | InaAs; dGs; InaTs; dTs; InaCs; |
| | | | | | | dCs; InaAs; dGs; InaA-Sup |
| 14 | Oligo14 | TGGTCTCATC | Internal | FXN | human | dTs; InaGs; dGs; InaTs; dCs; InaTs; |
| | | TAGAGAGCC | | | | dCs; InaAs; dTs; InaCs; dTs; |
| | | T | | | | InaAs; dGs; InaAs; dGs; InaAs; |
| | | | | | | dGs; InaCs; dCs; InaT-Sup |
| 15 | Oligo15 | CTCTGCTAGT | Internal | FXN | human | dCs; InaTs; dCs; InaTs; dGs; InaCs; |
| | | CTTTCATAG | | | | dTs; InaAs; dGs; InaTs; dCs; |
| | | G | | | | InaTs; dTs; InaTs; dCs; InaAs; |
| | | | | | | dTs; InaAs; dGs; InaG-Sup |
| 16 | Oligo16 | GCTAAAGAG | Internal | FXN | human | dGs; InaCs; dTs; InaAs; dAs; InaAs; |
| | | TCCAGCGTTT | | | | dGs; InaAs; dGs; InaTs; dCs; |
| | | C | | | | InaCs; dAs; InaGs; dCs; InaGs; |
| | | | | | | dTs; InaTs; dTs; InaC-Sup |
| 17 | Oligo17 | GCAAGGTCT | Internal | FXN | human | dGs; InaCs; dAs; InaAs; dGs; InaGs; |
| | | TCAAAAAAC | | | | dTs; InaCs; dTs; InaTs; dCs; |
| | | TCT | | | | InaAs; dAs; InaAs; dAs; InaAs; |
| | | | | | | dAs; InaCs; dTs; InaCs; dT- |
| | | | | | | Sup |
| 18 | Oligo18 | CTCAAACGT | Internal | FXN | human | dCs; InaTs; dCs; InaAs; dAs; InaAs; |
| | | GTATGGCTT | | | | dCs; InaGs; dTs; InaGs; dTs; |
| | | GTCT | | | | InaAs; dTs; InaGs; dGs; InaCs; |
| | | | | | | dTs; InaTs; dGs; InaTs; dCs; InaT- |
| | | | | | | Sup |
| 19 | Oligo19 | CCCAAAGGA | Internal | FXN | human | dCs; InaCs; dCs; InaAs; dAs; InaAs; |
| | | GACATCATA | | | | dGs; InaGs; dAs; InaGs; dAs; |
| | | GTC | | | | InaCs; dAs; InaTs; dCs; InaAs; |
| | | | | | | dTs; InaAs; dGs; InaTs; dC- |
| | | | | | | Sup |
| 20 | Oligo20 | CAGTTTGAC | Internal | FXN | human | dCs; InaAs; dGs; InaTs; dTs; InaTs; |
| | | AGTTAAGAC | | | | dGs; InaAs; dCs; InaAs; dGs; |
| | | ACCACT | | | | InaTs; dTs; InaAs; dAs; InaGs; |
| | | | | | | dAs; InaCs; dAs; InaCs; dCs; InaAs; |
| | | | | | | dCs; InaT-Sup |
| 21 | Oligo2l | ATAGGTTCC | Internal | FXN | human | dAs; InaTs; dAs; InaGs; dGs; InaTs; |
| | | TAGATCTCC | | | | dTs; InaCs; dCs; InaTs; dAs; |
| | | ACC | | | | InaGs; dAs; InaTs; dCs; InaTs; |
| | | | | | | dCs; InaCs; dAs; InaCs; dC- |
| | | | | | | Sup |
| 22 | Oligo22 | GGCGTCTGC | Internal | FXN | human | dGs; InaGs; dCs; InaGs; dTs; InaCs; |
| | | TTGTTGATCA | | | | dTs; InaGs; dCs; InaTs; dTs; |
| | | C | | | | InaGs; dTs; InaTs; dGs; InaAs; |
| | | | | | | dTs; InaCs; dAs; InaC-Sup |
| 23 | Oligo23 | AAGATAGCC | Internal | FXN | human | dAs; InaAs; dGs; InaAs; dTs; InaAs; |
| | | AGATTTGCTT | | | | dGs; InaCs; dCs; InaAs; dGs; |
| | | GTTT | | | | InaAs; dTs; InaTs; dTs; InaGs; |
| | | | | | | dCs; InaTs; dTs; InaGs; dTs; |
| | | | | | | InaTs; dT-Sup |
| 24 | Oligo24 | GGTCCACTA | Internal | FXN | human | dGs; InaGs; dTs; InaCs; dCs; InaAs; |
| | | CATACCTGG | | | | dCs; InaTs; dAs; InaCs; dAs; |
| | | ATGGAG | | | | InaTs; dAs; InaCs; dCs; InaTs; |
| | | | | | | dGs; InaGs; dAs; InaTs; dGs; |
| | | | | | | InaGs; dAs; InaG-Sup |
| 25 | Oligo25 | CCCAGTCCA | Internal | FXN | human | dCs; InaCs; dCs; InaAs; dGs; InaTs; |
| | | GTCATAACG | | | | dCs; InaCs; dAs; InaGs; dTs; |
| | | CTT | | | | InaCs; dAs; InaTs; dAs; InaAs; |
| | | | | | | dCs; InaGs; dCs; InaTs; dT- |
| | | | | | | Sup |
| 26 | Oligo26 | CGTGGGAGT | Internal | FXN | human | dCs; InaGs; dTs; InaGs; dGs; InaGs; |
| | | ACACCCAGT | | | | dAs; InaGs; dTs; InaAs; dCs; |
| | | TTTT | | | | InaAs; dCs; InaCs; dCs; InaAs; |
| | | | | | | dGs; InaTs; dTs; InaTs; dTs; InaT- |
| | | | | | | Sup |
| 27 | Oligo27 | CATGGAGGG | Internal | FXN | human | dCs; InaAs; dTs; InaGs; dGs; InaAs; |
| | | ACACGCCGT | | | | dGs; InaGs; dGs; InaAs; dCs; |
| | | | | | | InaAs; dCs; InaGs; dCs; InaCs; |
| | | | | | | dGs; InaT-Sup |
| 28 | Oligo28 | GTGAGCTCT | Internal | FXN | human | dGs; InaTs; dGs; InaAs; dGs; InaCs; |
| | | GCGGCCAGC | | | | dTs; InaCs; dTs; InaGs; dCs; |
| | | AGCT | | | | InaGs; dGs; InaCs; dCs; InaAs; |
| | | | | | | dGs; InaCs; dAs; InaGs; dCs; |
| | | | | | | InaT-Sup |
| 29 | Oligo29 | AGTTTGGTTT | Internal | FXN | human | dAs; InaGs; dTs; InaTs; dTs; InaGs; |
| | | TTAAGGCTTT | | | | dGs; InaTs; dTs; InaTs; dTs; |
| | | A | | | | InaTs; dAs; InaAs; dGs; InaGs; |
| | | | | | | dCs; InaTs; dTs; InaTs; dA-Sup |
| 30 | Oligo30 | TAGGCCAAG | Internal | FXN | human | dTs; InaAs; dGs; InaGs; dCs; InaCs; |
| | | GAAGACAAG | | | | dAs; InaAs; dGs; InaGs; dAs; |
| | | TCC | | | | InaAs; dGs; InaAs; dCs; InaAs; |
| | | | | | | dAs; InaGs; dTs; InaCs; dC- |
| | | | | | | Sup |
| 31 | Oligo31 | TCAAGCATC | Internal | FXN | human | dTs; InaCs; dAs; InaAs; dGs; InaCs; |
| | | TTTTCCGGA | | | | dAs; InaTs; dCs; InaTs; dTs; |
| | | A | | | | InaTs; dTs; InaCs; dCs; InaGs; |
| | | | | | | dGs; InaAs; dA-Sup |
| 32 | Oligo32 | TCCTTAAAAC | Internal | FXN | human | dTs; InaCs; dCs; InaTs; dTs; InaAs; |
| | | GGGGCTGG | | | | dAs; InaAs; dAs; InaCs; dGs; |
| | | GCA | | | | InaGs; dGs; InaGs; dCs; InaTs; |
| | | | | | | dGs; InaGs; dGs; InaCs; dA- |
| | | | | | | Sup |
| 33 | Oligo33 | TTGGCCTGA | Internal | FXN | human | dTs; InaTs; dGs; InaGs; dCs; InaCs; |
| | | TAGCTTTTAA | | | | dTs; InaGs; dAs; InaTs; dAs; |
| | | TG | | | | InaGs; dCs; InaTs; dTs; InaTs; |
| | | | | | | dTs; InaAs; dAs; InaTs; dG- |
| | | | | | | Sup |
| 34 | Oligo34 | CCTCAGCTG | Internal | FXN | human | dCs; InaCs; dTs; InaCs; dAs; InaGs; |
| | | CATAATGAA | | | | dCs; InaTs; dGs; InaCs; dAs; |
| | | GCTGGGGTC | | | | InaTs; dAs; InaAs; dTs; InaGs; |
| | | | | | | dAs; InaAs; dGs; InaCs; dTs; InaGs; |
| | | | | | | dGs; InaGs; dGs; InaTs; dC- |
| | | | | | | Sup |
| 35 | Oligo35 | AACAACAAC | Internal | FXN | human | dAs; InaAs; dCs; InaAs; dAs; InaCs; |
| | | AACAACAAA | | | | dAs; InaAs; dCs; InaAs; dAs; |
| | | AAACAGA | | | | InaCs; dAs; InaAs; dCs; InaAs; |
| | | | | | | dAs; InaAs; dAs; InaAs; dAs; |
| | | | | | | InaCs; dAs; InaGs; dA-Sup |
| 36 | Oligo36 | CCTCAAAAG | Internal | FXN | human | dCs; InaCs; dTs; InaCs; dAs; InaAs; |
| | | CAGGAATAA | | | | dAs; InaAs; dGs; InaCs; dAs; |
| | | AAAAAATA | | | | InaGs; dGs; InaAs; dAs; InaTs; |
| | | | | | | dAs; InaAs; dAs; InaAs; dAs; |
| | | | | | | InaAs; dAs; InaAs; dTs; InaA-Sup |
| 37 | Oligo37 | GCTGTGACA | Internal | FXN | human | dGs; InaCs; dTs; InaGs; dTs; InaGs; |
| | | CATAGCCCA | | | | dAs; InaCs; dAs; InaCs; dAs; |
| | | ACTGT | | | | InaTs; dAs; InaGs; dCs; InaCs; |
| | | | | | | dCs; InaAs; dAs; InaCs; dTs; |
| | | | | | | InaGs; dT-Sup |
| 38 | Oligo38 | GGAGGCAAC | Internal | FXN | human | dGs; InaGs; dAs; InaGs; dGs; InaCs; |
| | | ACATTCTTTC | | | | dAs; InaAs; dCs; InaAs; dCs; |
| | | TACAGA | | | | InaAs; dTs; InaTs; dCs; InaTs; |
| | | | | | | dTs; InaTs; dCs; InaTs; dAs; InaCs; |
| | | | | | | dAs; InaGs; dA-Sup |
| 39 | Oligo39 | CTATTAATAT | Intron | FXN | human | dCs; InaTs; dAs; InaTs; dTs; InaAs; |
| | | TACTG | | | | dAs; InaTs; dAs; InaTs; dTs; |
| | | | | | | InaAs; dCs; InaTs; dG-Sup |
| 40 | Oligo40 | CATTATGTGT | Intron | FXN | human | dCs; InaAs; dTs; InaTs; dAs; InaTs; |
| | | ATGTAT | | | | dGs; InaTs; dGs; InaTs; dAs; |
| | | | | | | InaTs; dGs; InaTs; dAs; InaT- |
| | | | | | | Sup |
| 41 | Oligo41 | TTTATCTATG | Intron | FXN | human | dTs; InaTs; dTs; InaAs; dTs; InaCs; |
| | | TTATT | | | | dTs; InaAs; dTs; InaGs; dTs; |
| | | | | | | InaTs; dAs; InaTs; dT-Sup |
| 42 | Oligo42 | CTAATTTGA | Intron | FXN | human | dCs; InaTs; dAs; InaAs; dTs; InaTs; |
| | | AGTTCT | | | | dTs; InaGs; dAs; InaAs; dGs; |
| | | | | | | InaTs; dTs; InaCs; dT-Sup |
| 43 | Oligo43 | TTCGAACTT | Exon | FXN | human | dTs; InaTs; dCs; InaGs; dAs; InaAs; |
| | | GCGCGG | Spanning | | | dCs; InaTs; dTs; InaGs; dCs; |
| | | | | | | InaGs; dCs; InaGs; dG-Sup |
| 44 | Oligo44 | TAGAGAGCC | Exon | FXN | human | dTs; InaAs; dGs; InaAs; dGs; InaAs; |
| | | TGGGT | Spanning | | | dGs; InaCs; dCs; InaTs; dGs; |
| | | | | | | InaGs; dGs; InaT-Sup |
| 45 | Oligo45 | ACACCACTC | Exon | FXN | human | dAs; InaCs; dAs; InaCs; dCs; InaAs; |
| | | CCAAAG | Spanning | | | dCs; InaTs; dCs; InaCs; dCs; |
| | | | | | | InaAs; dAs; InaAs; dG-Sup |
| 46 | Oligo46 | AGGTCCACT | Exon | FXN | human | dAs; InaGs; dGs; InaTs; dCs; InaCs; |
| | | ACATAC | Spanning | | | dAs; InaCs; dTs; InaAs; dCs; |
| | | | | | | InaAs; dTs; InaAs; dC-Sup |
| 47 | Oligo47 | CGTTAACCT | Exon | FXN | human | dCs; InaGs; dTs; InaTs; dAs; InaAs; |
| | | GGATGG | Spanning | | | dCs; InaCs; dTs; InaGs; dGs; |
| | | | | | | InaAs; dTs; InaGs; dG-Sup |
| 80 | Oligo81 | AAAGCCTTA | Antisense | FXN | human | dAs; InaAs; dAs; InaGs; dCs; InaCs; |
| | | AAAACC | | | | dTs; InaTs; dAs; InaAs; dAs; |
| | | | | | | InaAs; dAs; InaCs; dC-Sup |
| 81 | Oligo82 | TCAGGCCAA | Antisense | FXN | human | dTs; InaCs; dAs; InaGs; dGs; InaCs; |
| | | GACCCC | | | | dCs; InaAs; dAs; InaGs; dAs; |
| | | | | | | InaCs; dCs; InaCs; dC-Sup |
| 82 | Oligo83 | CCCAGCTTC | Antisense | FXN | human | dCs; InaCs; dCs; InaAs; dGs; InaCs; |
| | | ATTATG | | | | dTs; InaTs; dCs; InaAs; dTs; |
| | | | | | | InaTs; dAs; InaTs; dG-Sup |
| 83 | Oligo84 | AATGTGTTG | Antisense | FXN | human | dAs; InaAs; dTs; InaGs; dTs; InaGs; |
| | | CCTCCT | | | | dTs; InaTs; dGs; InaCs; dCs; |
| | | | | | | InaTs; dCs; InaCs; dT-Sup |
| 84 | Oligo85 | AAAAAGCAA | Antisense | FXN | human | dAs; InaAs; dAs; InaAs; dAs; InaGs; |
| | | AATAAT | | | | dCs; InaAs; dAs; InaAs; dAs; |
| | | | | | | InaTs; dAs; InaAs; dT-Sup |
| 85 | Oligo86 | CCAGGAGGG | Antisense | FXN | human | dCs; InaCs; dAs; InaGs; dGs; InaAs; |
| | | AAAATG | | | | dGs; InaGs; dGs; InaAs; dAs; |
| | | | | | | InaAs; dAs; InaTs; dG-Sup |
| 86 | Oligo87 | TAAAGGGTA | Antisense | FXN | human | dTs; InaAs; dAs; InaAs; dGs; InaGs; |
| | | GCCTAC | | | | dGs; InaTs; dAs; InaGs; dCs; |
| | | | | | | InaCs; dTs; InaAs; dC-Sup |
| 87 | Oligo88 | AAAGCAACA | Antisense | FXN | human | dAs; InaAs; dAs; InaGs; dCs; InaAs; |
| | | AGCCTC | | | | dAs; InaCs; dAs; InaAs; dGs; |
| | | | | | | InaCs; dCs; InaTs; dC-Sup |
| 88 | Oligo89 | ATAACATCA | Antisense | FXN | human | dAs; InaTs; dAs; InaAs; dCs; InaAs; |
| | | TACATG | | | | dTs; InaCs; dAs; InaTs; dAs; |
| | | | | | | InaCs; dAs; InaTs; dG-Sup |
| 89 | Oligo90 | GATACTATCT | Antisense | FXN | human | dGs; InaAs; dTs; InaAs; dCs; InaTs; |
| | | TCCTC | | | | dAs; InaTs; dCs; InaTs; dTs; |
| | | | | | | InaCs; dCs; InaTs; dC-Sup |
| 77 | Oligo91 | ATGGGGGAC | Antisense | FXN | human | dAs; InaTs; dGs; InaGs; dGs; InaGs; |
| | | GGGGCA | | | | dGs; InaAs; dCs; InaGs; dGs; |
| | | | | | | InaGs; dGs; InaCs; dA-Sup |
| 78 | Oligo92 | GGTTGAGAC | Antisense | FXN | human | dGs; InaGs; dTs; InaTs; dGs; InaAs; |
| | | TGGGTG | | | | dGs; InaAs; dCs; InaTs; dGs; |
| | | | | | | InaGs; dGs; InaTs; dG-Sup |
| 90 | Oligo93 | AGACTGAAG | Antisense | FXN | human | dAs; InaGs; dAs; InaCs; dTs; InaGs; |
| | | AGGTGC | | | | dAs; InaAs; dGs; InaAs; dGs; |
| | | | | | | InaGs; dTs; InaGs; dC-Sup |
| 91 | Oligo94 | CGGGACGGC | Antisense | FXN | human | dCs; InaGs; dGs; InaGs; dAs; InaCs; |
| | | TGTGTT | | | | dGs; InaGs; dCs; InaTs; dGs; |
| | | | | | | InaTs; dGs; InaTs; dT-Sup |
| 92 | Oligo95 | TCTGTGTGG | Antisense | FXN | human | dTs; InaCs; dTs; InaGs; dTs; InaGs; |
| | | GCAGCA | | | | dTs; InaGs; dGs; InaGs; dCs; |
| | | | | | | InaAs; dGs; InaCs; dA-Sup |
| 93 | Oligo96 | AAAGCCTTA | Antisense | FXN | human | InaAs; InaAs; InaAs; dGs; dCs; |
| | | AAAACC | | | | dCs; dTs; dTs; dAs; dAs; dAs; |
| | | | | | | dAs; InaAs; InaCs; InaC-Sup |
| 94 | Oligo97 | TCAGGCCAA | Antisense | FXN | human | InaTs; InaCs; InaAs; dGs; dGs; |
| | | GACCCC | | | | dCs; dCs; dAs; dAs; dGs; dAs; |
| | | | | | | dCs; InaCs; InaCs; InaC-Sup |
| 95 | Oligo98 | CCCAGCTTC | Antisense | FXN | human | InaCs; InaCs; InaCs; dAs; dGs; |
| | | ATTATG | | | | dCs; dTs; dTs; dCs; dAs; dTs; dTs; |
| | | | | | | InaAs; InaTs; InaG-Sup |
| 96 | Oligo99 | AATGTGTTG | Antisense | FXN | human | InaAs; InaAs; InaTs; dGs; dTs; |
| | | CCTCCT | | | | dGs; dTs; dTs; dGs; dCs; dCs; dTs; |
| | | | | | | InaCs; InaCs; InaT-Sup |
| 97 | Oligo100 | AAAAAGCAA | Antisense | FXN | human | InaAs; InaAs; InaAs; dAs; dAs; |
| | | AATAAT | | | | dGs; dCs; dAs; dAs; dAs; dAs; dTs; |
| | | | | | | InaAs; InaAs; InaT-Sup |
| 98 | Oligo101 | CCAGGAGGG | Antisense | FXN | human | InaCs; InaCs; InaAs; dGs; dGs; |
| | | AAAATG | | | | dAs; dGs; dGs; dGs; dAs; dAs; dAs; |
| | | | | | | InaAs; InaTs; InaG-Sup |
| 99 | Oligo102 | TAAAGGGTA | Antisense | FXN | human | InaTs; InaAs; InaAs; dAs; dGs; |
| | | GCCTAC | | | | dGs; dGs; dTs; dAs; dGs; dCs; dCs; |
| | | | | | | InaTs; InaAs; InaC-Sup |
| 100 | Oligo103 | AAAGCAACA | Antisense | FXN | human | InaAs; InaAs; InaAs; dGs; dCs; |
| | | AGCCTC | | | | dAs; dAs; dCs; dAs; dAs; dGs; dCs; |
| | | | | | | InaCs; InaTs; InaC-Sup |
| 101 | Oligo104 | ATAACATCA | Antisense | FXN | human | InaAs; InaTs; InaAs; dAs; dCs; |
| | | TACATG | | | | dAs; dTs; dCs; dAs; dTs; dAs; dCs; |
| | | | | | | InaAs; InaTs; InaG-Sup |
| 102 | Oligo105 | GATACTATCT | Antisense | FXN | human | InaGs; InaAs; InaTs; dAs; dCs; |
| | | TCCTC | | | | dTs; dAs; dTs; dCs; dTs; dTs; dCs; |
| | | | | | | InaCs; InaTs; InaC-Sup |
| 103 | Oligo106 | ATGGGGGAC | Antisense | FXN | human | InaAs; InaTs; InaGs; dGs; dGs; |
| | | GGGGCA | | | | dGs; dGs; dAs; dCs; dGs; dGs; dGs; |
| | | | | | | InaGs; InaCs; InaA-Sup |
| 104 | Oligo107 | GGTTGAGAC | Antisense | FXN | human | InaGs; InaGs; InaTs; dTs; dGs; |
| | | TGGGTG | | | | dAs; dGs; dAs; dCs; dTs; dGs; dGs; |
| | | | | | | InaGs; InaTs; InaG-Sup |
| 105 | Oligo108 | AGACTGAAG | Antisense | FXN | human | InaAs; InaGs; InaAs; dCs; dTs; |
| | | AGGTGC | | | | dGs; dAs; dAs; dGs; dAs; dGs; dGs; |
| | | | | | | InaTs; InaGs; InaC-Sup |
| 106 | Oligo109 | CGGGACGGC | Antisense | FXN | human | InaCs; InaGs; InaGs; dGs; dAs; |
| | | TGTGTT | | | | dCs; dGs; dGs; dCs; dTs; dGs; dTs; |
| | | | | | | InaGs; InaTs; InaT-Sup |
| 107 | Oligo110 | TCTGTGTGG | Antisense | FXN | human | InaTs; InaCs; InaTs; dGs; dTs; dGs; |
| | | GCAGCA | | | | dTs; dGs; dGs; dGs; dCs; dA |
| | | | | | | s; InaGs; InaCs; InaA-Sup |
| 108 | Oligo111 | GAAGAAGAA | Antisense | FXN | human | InaGs; InaAs; InaAs; dGs; dAs; |
| | | GAAGAA | | | | dAs; dGs; dAs; dAs; dGs; dAs; dAs; |
| | | | | | | InaGs; InaAs; InaA-Sup |
| 109 | Oligo112 | TTCTTCTTCT | Antisense | FXN | human | InaTs; InaTs; InaCs; dTs; dTs; |
| | | TCTTC | | | | dCs; dTs; dTs; dCs; dTs; dTs; dCs; |
| | | | | | | InaTs; InaTs; InaC-Sup |

TABLE 4
Oligonucleotide Modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |

In Vitro Transfection of Cells with Oligonucleotides

Cells were seeded into each well of 24-well plates at a density of 25,000 cells per 500 uL and transfections are performed with Lipofectamine and the oligonucleotides. Control wells contained Lipofectamine alone. At time points post-transfection, approximately 200 uL of cell culture supernatants was stored at −80 C for ELISA and RNA was harvested from another aliquot of cells and quantitative PCR was carried out as outlined above. The percent induction of FXN mRNA expression by each oligonucleotide was determined by normalizing mRNA levels in the presence of the oligonucleotide to the mRNA levels in the presence of control (Lipofectamine alone).

### RESULTS

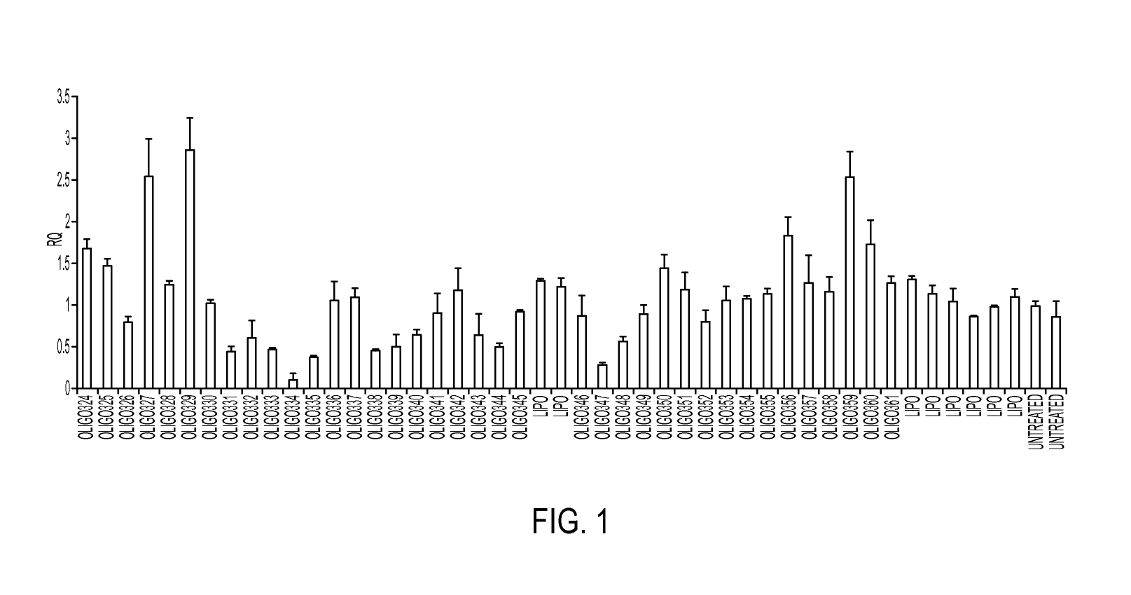

A screen of oligonucleotides complementary to a region of the sense strand of the human FXN DNA and/or complementary to a region of the human FXN mRNA were tested in a FRDA patient cell line to determine if the oligonucleotides were capable of upregulating FXN. Firstly, cell lines from FRDA patients were treated with mixmer oligonucleotides that were complementary to either an internal region (e.g., an intron or exon or intron/exon boundary) or a UTR region of the human FXN gene or the human FXN mRNA. A subset of the oligonucleotides were found to upregulate FXN mRNA, with oligos 327, 329 and 359 having the highest level of FXN mRNA upregulation (FIG. 1). These 3 oligos were complementary to a UTR region of human FXN.

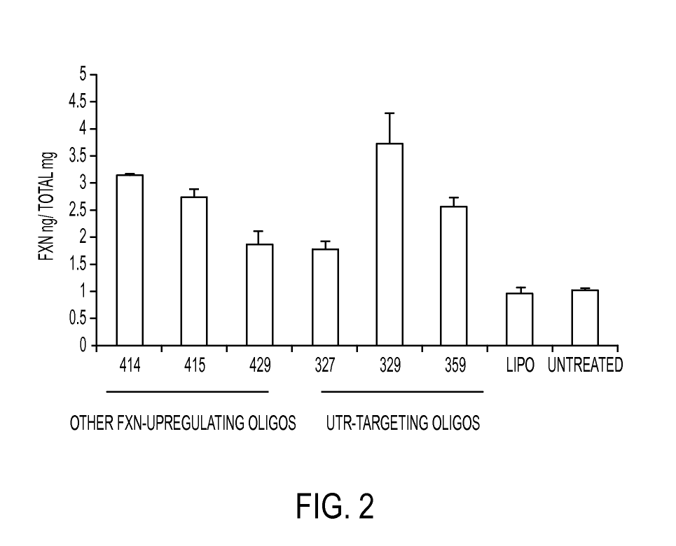

Next, oligonucleotides 327, 329 and 359, and other oligonucleotides shown to upregulated FXN mRNA (oligos 414, 415, and 429), were tested to see if the oligonucleotides could upregulate FXN protein levels. All tested oligos were shown to increase the level of FXN protein in a FRDA patient cell line (FIG. 2).

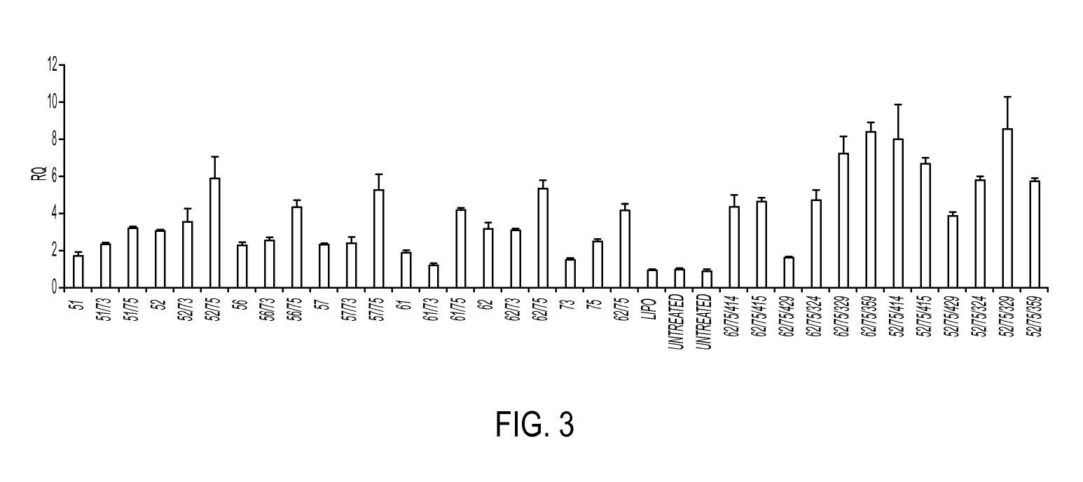

Finally, a combination of FXN RNA stabilizing oligonucleotides and oligos complementary to a region of the human FXN DNA or mRNA were tested in a FRDA patient cell line. It was found that several combinations of oligonucleotides increased FXN mRNA levels (FIG. 3).

TABLE 5
Oligos of FIG. 3

| SEQ | Oligo | | Targeting | Gene | | |
|---|---|---|---|---|---|---|
| ID NO | Name | Base Sequence | Region | Name | Organism | Formatted Sequence |
| 51 | 51 | CGCCCTCCAGCG | 5′-End | FXN | human | dCs; InaGs; dCs; InaCs; dCs; InaTs; |
| | | CTG | | | | dCs; InaCs; dAs; InaGs; dCs; InaGs; |
| | | | | | | dCs; InaTs; dG-Sup |
| 52 | 52 | CGCTCCGCCCTC | 5′-End | FXN | human | dCs; InaGs; dCs; InaTs; dCs; InaCs; |
| | | CAG | | | | dGs; InaCs; dCs; InaCs; dTs; InaCs; |
| | | | | | | dCs; InaAs; dG-Sup |
| 56 | 56 | CGCCCTCCAGCG | 5′-End | FXN | human | dCs; InaGs; dCs; InaCs; dCs; InaTs; |
| | | CTGCC | | | | dCs; InaCs; dAs; InaGs; dCs; InaGs; |
| | | | | | | dCs; InaTs; dGs; InaCs; dC-Sup |
| 57 | 57 | CGCTCCGCCCTC | 5′-End | FXN | human | dCs; InaGs; dCs; InaTs; dCs; InaCs; |
| | | CAGCC | | | | dGs; InaCs; dCs; InaCs; dTs; InaCs; |
| | | | | | | dCs; InaAs; dGs; InaCs; dC-Sup |
| 61 | 61 | CGCCCTCCAGCG | 5′-End | FXN | human | dCs; InaGs; dCs; InaCs; dCs; InaTs; |
| | | CTGGGAAACCTC | | | | dCs; InaCs; dAs; InaGs; dCs; InaGs; |
| | | | | | | dCs; InaTs; dGs; InaGs; dGs; dAs; dAs; |
| | | | | | | dAs; dCs; InaCs; dTs; InaC-Sup |
| 62 | 62 | CGCTCCGCCCTC | 5′-End | FXN | human | dCs; InaGs; dCs; InaTs; dCs; InaCs; |
| | | CAGCCAAAGGTC | | | | dGs; InaCs; dCs; InaCs; dTs; InaCs; |
| | | | | | | dCs; InaAs; dGs; InaCs; dCs; dAs; dAs; |
| | | | | | | dAs; dGs; InaGs; dTs; InaC-Sup |
| 73 | 73 | TTTTTGGGGTCTT | 3′-End | FXN | human | dTs; InaTs; dTs; InaTs; dTs; InaGs; |
| | | GGCCTGA | | | | dGs; InaGs; dGs; InaTs; dCs; InaTs; |
| | | | | | | dTs; InaGs; dGs; InaCs; dCs; InaTs; |
| | | | | | | dGs; InaA-Sup |
| 75 | 75 | TTTTTAGGAGGC | 3′-End | FXN | human | dTs; InaTs; dTs; InaTs; dTs; InaAs; |
| | | AACACATT | | | | dGs; InaGs; dAs; InaGs; dGs; InaCs; |
| | | | | | | dAs; InaAs; dCs; InaAs; dCs; InaAs; |
| | | | | | | dTs; InaT-Sup |

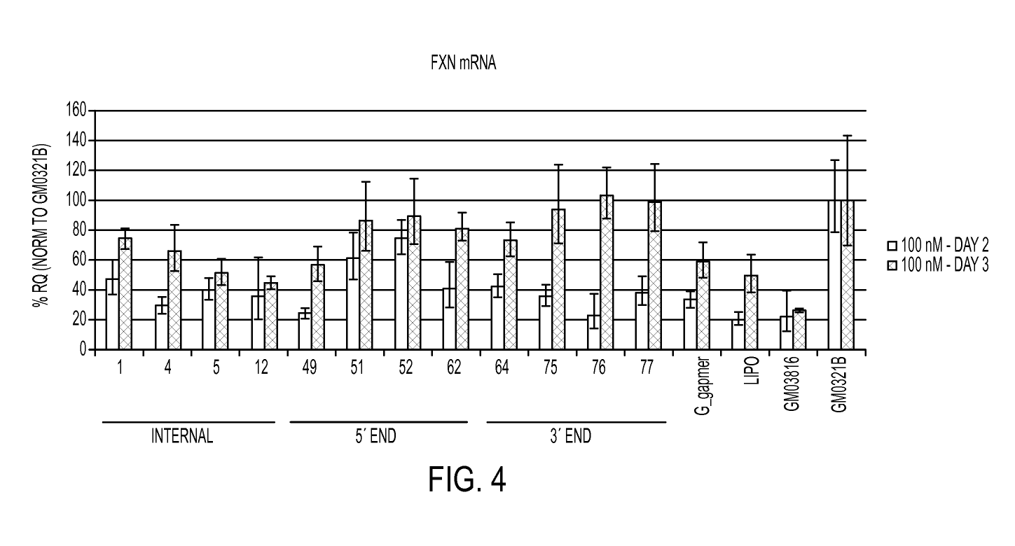

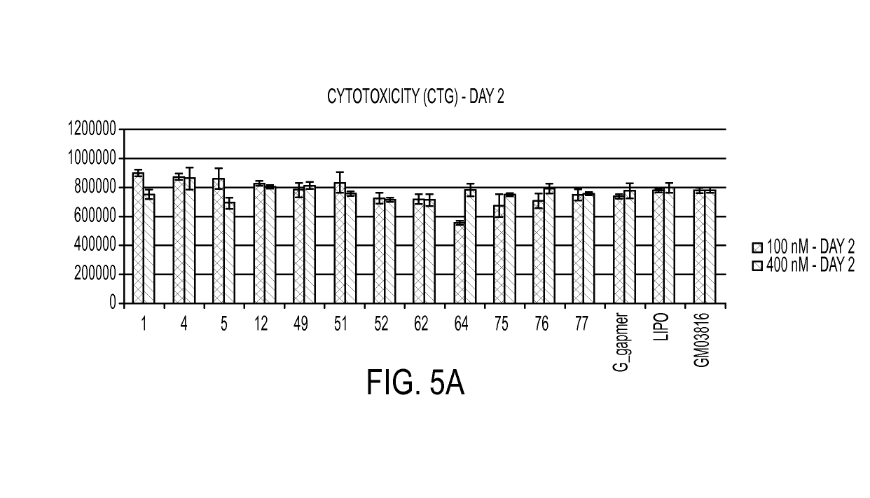

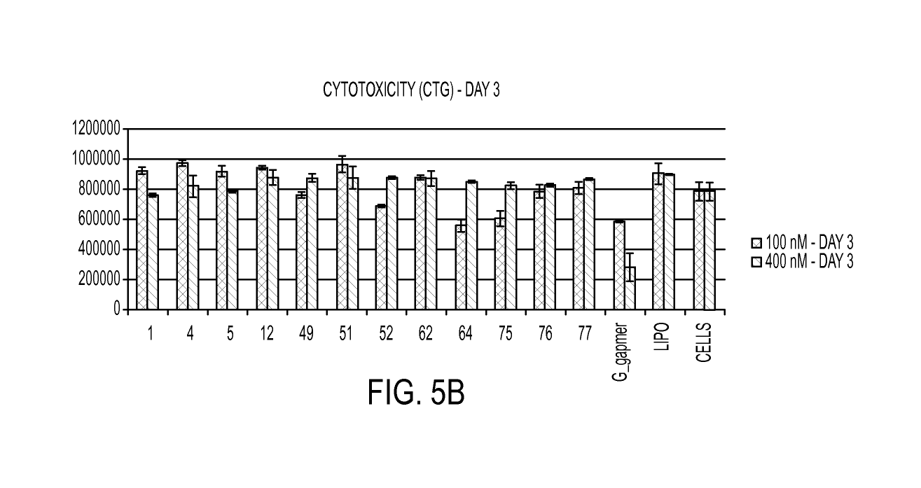

Other oligonucleotides complementary to a region of the human FXN DNA or mRNA (internal, 5′ end, or 3′ end) were also tested in a FRDA patient cell line. Several oligonucleotides showed an increase in FXN mRNA on at least day 3 post-transfection (FIG. 4). For a subset of the oligonucleotides tested, the steady-state mRNA levels of the oligos approached the levels of FXN mRNA in the GM0321B normal fibroblast cells. These oligos were found to not cause cytotoxicity to cells on days 2 and 3 post-transfection (FIG. 5).

These results indicate that oligonucleotides complementary to a region of a FXN target, used alone or in combination with other oligonucleotides that target a FXN target, are capable of upregulating FXN mRNA and protein levels and are not cytotoxic to cells.

### Example 2

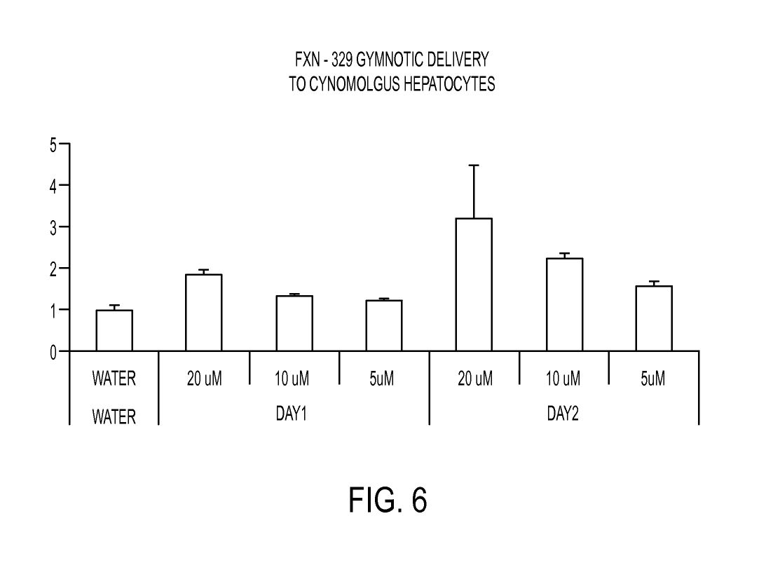

The FXN-329 oligo was delivered gymnotically into hepatocytes derived from Cyno (cynomolgus monkey). Treatment concentrations were 20 uM, 10 uM and 5 uM. FXN RNA measurements were taken at days 1 and 2 post treatment. Dose-responsive FXN mRNA upregulation was observed with oligo FXN-329 (FIG. 6).

Without further elaboration, it is believed that one skilled in the art can, based on the description provided herein, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method for increasing expression of Frataxin (FXN) in a cell, the method comprising:
delivering to a cell an oligonucleotide comprising at least 8 nucleotides of a nucleotide sequence as set forth in Table 2 or 3, thereby increasing FXN expression in the cell.

2. The method of claim 1, wherein prior to the step of delivering the cell has a higher level of histone H3 K27 or K9 methylation at the FXN gene compared with an appropriate control level of histone H3 K27 or K9 methylation.

3. The method of claim 1, wherein the cell comprises an FXN gene encoding in its first intron a GAA repeat of between 10-2000 units.

4. The method of claim 1, wherein the cell is in a subject having Friedreich's ataxia.

5. The method of claim 1, wherein the oligonucleotide is a single stranded oligonucleotide.

6. The method of claim 1, wherein the oligonucleotide comprises at least one modified internucleoside linkage.

7. The method of claim 1, wherein the oligonucleotide comprises at least one modified nucleotide.

8. The method of claim 1, wherein the oligonucleotide comprises at least one nucleotide comprising a 2′ O-methyl.

9. The method of claim 1, wherein the oligonucleotide comprises at least one ribonucleotide, at least one deoxyribonucleotide, at least one 2′-fluoro-deoxyribonucleotide or at least one bridged nucleotide.

10. The method of claim 9, wherein the bridged nucleotide is a LNA nucleotide, a cEt nucleotide or a ENA modified nucleotide.

11. The method of claim 1, wherein each nucleotide of the oligonucleotide is a LNA nucleotide.

12. The method claim 1, wherein the oligonucleotide is mixmer.

13. The method of claim 12, wherein the oligonucleotide comprises alternating deoxyribonucleotides and 2′-fluoro-deoxyribonucleotides, 2′-O-methyl nucleotides, or bridged nucleotides.

14. The method of claim 1, wherein the oligonucleotide comprises a sequence as set forth in Table 2 or Table 3.

15. The method of claim 1, wherein the oligonucleotide is 8 to 50 nucleotides in length.

16. The method of claim 14, wherein the oligonucleotide consists of a sequence as set forth in Table 2 or Table 3.

17. An oligonucleotide of 8 to 50 nucleotides in length comprising at least 8 consecutive nucleotides of a nucleotide sequence as set forth in Table 2 or 3.

18. The oligonucleotide of claim 17, wherein the oligonucleotide is a single stranded oligonucleotide.

19. The oligonucleotide of claim 17, wherein the oligonucleotide comprises at least one modified internucleoside linkage.

20. The oligonucleotide of claim 17, wherein the oligonucleotide comprises at least one modified nucleotide.

21. The oligonucleotide of claim 17, wherein at least one nucleotide comprises a 2′ O-methyl.

22. The oligonucleotide of claim 17, wherein the oligonucleotide comprises at least one ribonucleotide, at least one deoxyribonucleotide, at least one 2′-fluoro-deoxyribonucleotides or at least one bridged nucleotide.

23. The oligonucleotide of claim 22, wherein the bridged nucleotide is a LNA nucleotide, a cEt nucleotide or a ENA modified nucleotide.

24. The oligonucleotide of claim 17, wherein each nucleotide of the oligonucleotide is a LNA nucleotide.

25. The oligonucleotide of claim 17, wherein the oligonucleotide is mixmer.

26. The oligonucleotide of claim 25, wherein the nucleotides of the oligonucleotide comprise alternating deoxyribonucleotides and 2′-fluoro-deoxyribonucleotides, 2′-O-methyl nucleotides, or bridged nucleotides.

27. The oligonucleotide of claim 17, wherein the oligonucleotide comprises a sequence as set forth in Table 2 or Table 3.

28. The oligonucleotide of claim 17, wherein the oligonucleotide comprises a fragment of at least 8 nucleotides of a nucleotide sequence as set forth in Table 2 or 3.

29. The oligonucleotide of claim 17, wherein the oligonucleotide consists of a sequence as set forth in Table 2 or Table 3.

30. A composition comprising a plurality of oligonucleotides, wherein each of at least 75% of the oligonucleotides is an oligonucleotide of claim 17.

31. The composition of claim 30, wherein the oligonucleotides are complexed with a monovalent cation.

32. The composition of claim 30, wherein the oligonucleotides are in a lyophilized form.

33. The composition of claim 30, wherein the oligonucleotides are in an aqueous solution.

34. A composition comprising an oligonucleotide of claim 17 and a carrier.

35. A composition comprising an oligonucleotide of claim 17 in a buffered solution.

36. A composition of comprising an oligonucleotide of claim 17 conjugated to the carrier.

37. The composition of claim 36, wherein the carrier is a peptide.

38. The composition of claim 36, wherein the carrier is a steroid.

39. A pharmaceutical composition comprising an oligonucleotide of claim 17 and a pharmaceutically acceptable carrier.

40. A kit comprising a container housing the composition of claim 30.

41. A method of upregulating FXN in a subject in need thereof, the method comprising:
administering a therapeutically effective amount of an oligonucleotide of any claim 17.

42. The method of claim 41, wherein the subject is a subject having Friedrich's ataxia.
